(19) United States
(12) Patent Application Publication
(10) Pub. No.: US 2025/0110111 A1
(43) Pub. Date: Apr. 3, 2025

(54) DISPOSABLE SYSTEM FOR ANALYSIS OF HEMOSTATIC FUNCTION

(71) Applicant: HEMOSONICS, LLC, Charlottesville, VA (US)

(72) Inventors: Francesco Viola, Chapel Hill, NC (US); Timothy Higgins, Charlottesville, VA (US); Andrew Homyk, Charlottesville, VA (US); F. Scott Corey, IV, Baltimore, MD (US); Franklin F. Regan, IV, Charlottesville, VA (US); William F. Walker, Charlottesville, VA (US); David Bryant, Charlottesville, VA (US); Thomas Givens, Rougemont, VA (US); Cynthia Ann Lloyd, Durham, NC (US)

(21) Appl. No.: 18/828,641

(22) Filed: Sep. 9, 2024

Related U.S. Application Data
(63) Continuation of application No. 17/843,505, filed on Jun. 17, 2022, now Pat. No. 12,117,436.
Application No. 17/843,505 is a continuation of application No. 15/958,875, filed on Apr. 20, 2018, now Pat. No. 11,366,093.
(60) Provisional application No. 62/488,045, filed on Apr. 20, 2017.

(51) Int. Cl. G01N 33/49; B01L 3/00; G01N 33/86

(52) U.S. Cl. CPC G01N 33/4905; B01L 3/502; G01N 33/86; B01L 2300/0825; B01L 2300/0838

(57) ABSTRACT

A disposable system, in some embodiments, includes a multi-channel or multi-chamber test cartridge device configured to operate with a testing system for evaluation of hemostasis in a subject by in vitro evaluation of a test sample from the subject. The disposable system, in some embodiments, is configured to interrogate the test sample to evaluate clot stiffness, strength, or other mechanical properties of the test sample to assess the function of various physiological processes occur during coagulation and/or dissolution of the resulting clot.

### RELATED APPLICATIONS

This application is a continuation of U.S. Utility application Ser. No. 17/843,505 filed Jun. 17, 2022, which is a continuation of U.S. Utility application Ser. No. 15/958,875, filed Apr. 20, 2018, which claims priority to, and the benefit of, U.S. Provisional Appl. No. 62/488,045, filed Apr. 20, 2017, titled “Disposable System for Analysis of Hemostatic Function,” which is incorporated by reference herein in its entirety.

### GOVERNMENT LICENSE RIGHTS

The invention was made with government support under grant R44HL103030 awarded by National Heart Lung and Blood Institute. The government has certain rights in the invention.

### TECHNICAL FIELD

The present application relates to devices, systems and methods for evaluating hemostasis in a subject by preparation and analysis of a test sample from the subject.

### BACKGROUND

Hemostasis, the physiological control of bleeding, is a complex process incorporating the vasculature, platelets, coagulation factors, fibrinolytic proteins, and a variety of activators and inhibitors. Disruption of hemostasis plays a central role in the onset of myocardial infarction, stroke, pulmonary embolism, deep vein thrombosis and excessive bleeding. Consequently, in vitro diagnostics (IVD) are critically needed to quantify hemostatic function/dysfunction and direct appropriate treatment.

The process of coagulation is highly dependent, among other things, on the temperature at which it takes place. Under normal conditions, coagulation occurs at body temperature, which is optimal for the proper enzymatic action of the clotting factors in the cascade.

Preparation of the blood to be tested is also important, as the manner a blood sample is prepared prior to its evaluation can affect, for example, the actions of the vasculature components, platelets and other cellular components, coagulation factors, fibrinolytic components, and any inhibitor or activator of hemostasis.

### SUMMARY

Provided are devices, systems and methods for evaluation of hemostasis. For example, provided are disposable systems for analysis of hemostasis function. The disposable system, in some embodiments, includes a multi-channel or multi-chamber test cartridge device configured to operate with a testing system for evaluation of hemostasis in a subject by in vitro evaluation of a test sample from the subject. The disposable system, in some embodiments, is configured to interrogate the test sample to evaluate clot stiffness, strength, or other mechanical properties of the test sample to assess the function of various physiological processes occur during coagulation and/or dissolution of the resulting clot. The sample can include in whole, or in part, whole blood, plasma, platelet rich plasma, or platelet poor plasma. Furthermore, the sample can include one or more reagent (such as anticoagulants or anti-platelet drugs that might be present in the blood as collected), or one or more pharmacological treatment (such as in the case of heparin or low molecular weight heparin) or other inert components (such as polystyrene beads) that are added to the test sample before the cartridge device being used. The disposable system facilitates the point of care evaluation of hemostasis of a test sample that is robust (e.g., can be performed in non-laboratory environment), rapid (e.g., only to take a few minutes to perform), easy-to-use and provides clear results (e.g., that are direct to the functional components of hemostasis), and facilitates identification of exact hemostasis defects. The exemplified device automates one or more pre-measurement steps that minimizes sample manipulation steps required for the user, thereby improving test reproducibility and/or test quality. The disposable system, in some embodiments, includes a plurality of testing circuits each having a pathway defined by channels and chambers configured to prepare a test sample of blood for evaluation by a measurement device. In each testing circuit, a portion of the test sample is introduced to a reagent or combination of reagents specific to that testing circuit.

The disposable system, in some embodiments, is configured to condition the respective test samples prior to, during, and/or after the mixing with the reagent(s), to optimize the proper actions of applicable blood component and chemistry (e.g., vasculature components, platelets or other cellular components, coagulation factors, fibrinolytic components, and any other inhibitor or activator of hemostatic function, etc.) being evaluated.

In an aspect, an apparatus (e.g., a cartridge) is disclosed for the assessment of hemostasis. The apparatus includes a housing; an input port integrally formed with the housing that is structurally configured to establish fluidic communication and evacuate contents of a sample holding tube; and a first chamber in fluidic communication with the input port, the first chamber being configured to receive a sample contained in the sample holding tube and to condition the received sample to a desired temperature (e.g., a pre-defined temperature range) before the received sample is allowed to contact one or more reagents located in one or more fluidic circuits downstream to the first chamber, wherein each of the one or more fluidic circuits comprises i) a second chamber in fluidic communication with the first chamber that meters the sample in the first chamber into an aliquot, wherein the metered sample is introduced to a reagent, or a combination of reagents, (e.g., in the form of lyophilized reagent bead) located in a corresponding fluidic circuit (e.g., a reagent pocket) to form a mixed sample and ii) a testing chamber in fluidic communication with the second chamber, the testing chamber being structurally configured for interrogation by a measurement system configured to determine properties (e.g., mechanical properties or viscoelastic properties) of the mixed sample.

In some embodiments, at least one of the one or more fluidic circuits comprise one or more pockets (e.g., each configured to house a lyophilized reagent bead comprising a reagent, or a combination of reagents).

In some embodiments, at least one of the one or more fluidic circuits comprise one or more liquid-retaining pockets (e.g., each configured to house an assay, in liquid form, comprising the reagent, or a combination of reagents).

In some embodiments, at least one of the one or more fluidic circuits includes one or more lyophilized reagents that are located on one or more surfaces thereof (e.g., lyophilized on each of the surfaces; lyophilized as films placed on, or adhered to, one or more of the surfaces).

In some embodiments, at least one of the one or more fluidic circuits includes one or more reagents that are processed onto surfaces thereof (e.g., dried on the surfaces; spray coated on the surfaces; baked onto the surfaces).

In some embodiments, the input port is communicatively coupled to a pressure port, wherein pressure applied to the pressure port causes the evacuation of the contents of the sample holding tube through the input port to first chamber.

In some embodiments, the input port comprises a needle assembly.

In some embodiments, the needle assembly comprises the input port and a second port, wherein the second port is configured to vent a liquid or gas into the sample holding tube so as to promote evacuation of the contents therein. The input port, in some embodiments, is located (e.g., concentrically located) within a second port configured to vent a liquid or gas into the sample holding tube so as to cause evacuation of the contents of the sample holding tube.

In some embodiments, the input port comprises a luer lock configured to connect to the sample holding tube, wherein the sample holding tube is a syringe.

In some embodiments, the input port is communicatively coupled to a first pressure port, wherein pressure when applied to the first pressure port causes the evacuation of the contents of the sample holding tube through the input port to the first chamber.

In some embodiments, the first chamber is configured to mate with a corresponding thermal regulating system (e.g., heating/cooling system) of the measurement system to condition the received sample to, or near, the desired temperature.

In some embodiments, the shape and/or materials of the first chamber are optimized to facilitate thermal regulation (e.g., heating and/or cooling) of the sample to, or near, the desired temperature.

In some embodiments, the first chamber is configured to mate with a corresponding thermal regulating surface of a sub-system component of the measurement system to condition the received sample to, or near, the desired temperature. In some embodiments, a channel portion of the one or more fluidic circuits is configured to mate with a corresponding heating/cooling system of the measurement system to condition the received sample to, or near, the desired temperature.

In some embodiments, a channel portion of the one or more fluidic circuits is configured to mate with a corresponding thermal regulating system of the measurement system to condition the received sample to the desired temperature. In some embodiments, the first chamber and/or the channel portion of the one or more fluidic circuits is in physical proximity (e.g., physical contact or near contact) with a sensor configured to measure a temperature of the sample received in the first chamber.

In some embodiments, the sensor is selected from group consisting of a thermistor, a thermocouple, and an optical sensor (e.g., an IR sensor).

In some embodiments, the apparatus includes a first pressure port in fluidic communication with the first chamber, the first pressure port being configured to receive negative or differential pressure (e.g., for filling the first chamber); and a filter positioned within the first pressure port in at least one of the fluidic circuits (e.g., such that the filter is clogged by the sample received in the first chamber when the first chamber is full). The filter, in some embodiments, is configured to allow air to move through the first pressure port but prevent fluid from moving there through.

In some embodiments, the apparatus includes a first pressure port configured to receive negative or differential pressure for filling the first chamber; and a first fluidic pathway extending from the first pressure port to the first chamber, wherein the filter is positioned within the first pressure port.

In some embodiments, for each of the one or more fluidic circuits, the fluidic communication between the first chamber and the second chamber is through a second fluid pathway originating from a side of the first chamber (e.g., a side wall, a bottom wall, and etc.) (e.g., such that bubbles present in the received sample are trapped away from the second chamber).

In some embodiments, each of the one or more fluidic circuits comprises a third fluid pathway in fluidic communication with the second chamber, wherein the third fluidic pathway leads a second pressure port configured receive negative or differential pressure for filling the second chamber.

In some embodiments, the second pressure port has a second filter therein, wherein the second filter is configured to clog when the second chamber is filled.

In some embodiments, the apparatus includes one or more fluidic pathways in fluidic communication with the second pressure port for all of the one or more fluidic circuits, wherein the one or more fluidic pathways are configured to provide the negative pressure to the second pressure port for all of the one or more fluidic circuits.

In some embodiments, for each of the one or more fluidic circuits, the second chamber is in fluidic communication with a vent port, wherein the vent port is configured to be closed while the sample is metered into the aliquot in the second chamber and further configured to be open to atmospheric pressure after the sample is metered into the aliquot in the second chamber.

In some embodiments, each of the one or more fluidic circuits comprises a third set of fluid pathways in fluidic communication between a respective second chamber (e.g., metering chamber) and test chamber, wherein a portion of third set of fluid pathways are arranged as a serpentine-shaped conduit or channel.

In some embodiments, each of the one or more fluidic circuits further comprises a serpentine reservoir between the testing chamber and the second chamber.

In some embodiments, the metered sample is alternatively directed through portions of the one or more fluidic circuits to facilitate mixing of the metered sample and the reagent, or a combination of reagents.

In some embodiments, the metered sample is alternatively and multiplicatively directed, for each of the one or more fluidic circuits, between a first position (e.g., the second chamber) in a fluidic circuit a second position (e.g., a position in the serpentine reservoir) in the fluidic circuit.

In some embodiments, each of the one or more fluidic circuits further comprises a third pressure port in fluidic communication with the second chamber and the testing chamber, the third pressure port configured to receive negative or differential pressure (e.g., for drawing the aliquot from the second chamber to the testing chamber), wherein the third pressure port is further configured to alternately receive alternating pressure, e.g., for alternately drawing the aliquot from the second chamber along the serpentine reservoir and pushing the aliquot through the serpentine reservoir to the second chamber.

In some embodiments, the serpentine reservoir includes an optical detection zone to facilitate optical detection of the metered sample in the serpentine reservoir or a location of the sample in the serpentine reservoir.

In some embodiments, each of the one or more fluidic circuits further comprises a mixing pathway between the testing chamber and the second chamber, the mixing pathway comprising one or more ferromagnetic beads or bars therein.

In some embodiments, at least one of the one or more fluidic circuits comprises one or more quality testing portals.

In some embodiments, the one or more quality testing portals is configured to be sensed optically, wherein the quality testing port is transparent.

In some embodiments, the one or more quality testing portals is configured to be sensed electrically, wherein quality testing port comprises one or more sensing electrodes.

In some embodiments, the one or more quality testing ports is configured to be sampled for characteristics for the metered sample (e.g., for pressure, presence of flow, flow rate, temperature).

In some embodiments, for each of the one or more fluidic circuits, the testing chamber comprises a mechanism to couple energy into the testing chamber to perform the measurements such as in the case of a lens configured to direct ultrasonic pulses into to the testing chamber.

In another aspect, an apparatus is disclosed for the assessment of hemostasis, the apparatus comprising: a housing; an input port integrally formed with the housing that is structurally capable of establishing fluidic communication with, and evacuating contents of, a sample holding tube; a first chamber that is in fluidic communication with the input port that receives the sample contained in the evacuated tube and whereby the sample temperature is adjusted to a desired temperature before the sample contacting one or more reagents; one or more second chambers that are in fluidic communication with the first chamber, the one or more second chamber being configured to meter the sample in the first chamber into one or more aliquots; one or more reagent pockets each filled with one or more lyophilized reagent bead that are in fluidic communication with each of the aliquot chambers and permits the sample present in each aliquot to be mixed with said one or more reagent beads; and one or more testing chambers that are in fluidic communications with the aliquot chambers and that are structurally capable of being interrogated to determine the sample viscoelastic properties after such sample has been mixed with the one or more reagents.

In some embodiments, the reagent, or combination of reagents, located in the one or more fluidic circuits includes an intrinsic pathway activator (e.g., kaolin, celite, glass, ellagic acid, micronized silica, Hageman factor, etc.) or a combination therewith.

In some embodiments, the reagent, or combination of reagents, located in the one or more fluidic circuits includes an extrinsic pathway activator (e.g., tissue factor, recombinant tissue factor, thromboplastin, etc.) or a combination therewith.

In some embodiments, the reagent, or combination of reagents, located in the one or more fluidic circuits includes a coagulation activator (e.g., thrombin, factor Xa, reptilase, ecarin, Russell's viper venom or other snake venoms, etc.) or a combination therewith.

In some embodiments, the reagent, or combination of reagents, located in the one or more fluidic circuits includes a platelet activator or platelet inhibitor (e.g., GPIIb/IIIa inhibitors (e.g., abciximab, eptifibatide, tirofiban, roxifiban, orbofiban), cytochalasin D, blebbistatin, PAR1 inhibitors, PAR4 inhibitors, glycoprotein IB inhibitors, TRAP, ADP, arachidonic acid, ADP inhibitors, non-steroidal anti-inflammatory drugs, platelet activating factor, ristocetin, epinephrine, etc.) or a combination therewith.

In some embodiments, the reagent, or combination of reagents, located in the one or more fluidic circuits includes a fibrinolytic functions activator or inhibitor (e.g., tPA, uKA, streptokinase, TAFIa, plasmin/plasminogen, aprotinin, epsilon-aminocaproic acid, tranexamic acid, plasminogen activator inhibitor 1 (PAI1), α2-antiplasmin (α2-AP), or plasmin-antiplasmin complexes, carboxypeptidase inhibitor) or a combination therewith.

In some embodiments, the reagent, or combination of reagents, located in the one or more fluidic circuits includes FXIIIa inhibitors or a combination therewith.

In some embodiments, the reagent, or combination of reagents, located in the one or more fluidic circuit includes thrombomodulin or a combination therewith.

In some embodiments, the reagent, or combination of reagents, located in the one or more fluidic circuit includes low molecular weight heparin or a combination therewith.

In some embodiments, the reagent, or combination of reagents, located in the one or more fluidic circuits includes Hexadimethrine bromide (polybrene) or a combination therewith.

In some embodiments, the reagent, or combination of reagents, located in the one or more fluidic circuits includes heparin or a combination therewith.

In some embodiments, the reagent, or combination of reagents, located in the one or more fluidic circuits includes corn trypsin inhibitor or a combination therewith.

In some embodiments, the reagent, or combination of reagents, located in the one or more fluidic circuits includes adenosine or a combination therewith.

In some embodiments, the reagent, or combination of reagents, located in the one or more fluidic circuits includes GPRP (Gly-Pro-Arg-Pro) or a combination therewith.

In some embodiments, the reagent, or combination of reagents, located in the one or more fluidic circuits includes calcium or a combination therewith.

In some embodiments, the reagent, or combination of reagents, located in the one or more fluidic circuits includes fibronectin or a combination therewith.

In some embodiments, the reagent, or combination of reagents, located in the one or more fluidic circuits includes collagen or a combination therewith.

In some embodiments, the reagent, or combination of reagents, located in the one or more fluidic circuits includes an immuno-detection reagent or a combination therewith.

In some embodiments, the reagent, or combination of reagents, located in the one or more fluidic circuits includes heparinase I or a combination therewith.

In some embodiments, the reagent, or combination of reagents, located in the one or more fluidic circuits includes endothelial cells or activated endothelial cells.

In some embodiments, the measurement system is selected from the group consisting of a sonorheometry-based system, thromboelastography-based system, a thromboelastometry-based system, an optical-based system, a fluorescence-based system, a colorimetric-based system, an aggregometry-based system, a resonance-based system, and an electrical impedance-based system.

In another aspect, a method is disclosed of mixing a sample with one or more reagents in an apparatus (e.g., a cartridge) and testing the mixed sample for the assessment of hemostasis. The method includes receiving a plurality of metered samples of from a plurality of metering chambers that received test fluid from a sample holding tube (e.g., via a mechanical coupling that connects the apparatus to the sample holding tube or via an opening to which sample from the sample holding tube is placed); alternately and multiplicatively flowing each of the aliquots until the aliquot is mixed with a reagent, or a combination of reagents, to form a mixed aliquot, wherein the at least one aliquot alternately and cyclically flowed i) in a first direction from the metering chamber through one or more reagent pocket, with the one or more reagents therein (e.g., lyophilized reagent bead), and along a serpentine pathway in communication with the metering chamber until at least a portion of the aliquot reaches a detection zone located in, or after, the serpentine pathway and ii) in a second direction from the detection zone reversed to the first direction through at least a portion of the serpentine pathway toward the metering chamber until a trigger event; and driving the mixed aliquot in a testing chamber in fluidic communication with the metering chamber, wherein the testing chamber is structurally configured for interrogation by a measurement system configured to determine properties (e.g., mechanical properties or viscoelastic properties) of the mixed aliquot, and wherein an interrogation of the testing chamber is performed with the mixed aliquot located therein.

In some embodiments, the method includes receiving the fluid in a first chamber configured to substantially adjust the temperature of the test sample toward body temperature or other desired temperatures, wherein the metered sample received in the metering chamber is received from the first chamber.

In some embodiments, the test fluid is moved into the first chamber in response to an applied pressure that is applied by, or generated from, the measurement system.

In some embodiments, the method includes conditioning the test fluid in the first chamber to, or substantially near, a desired temperature, wherein the test fluid is mixed with the one or more reagents following exit from the first chamber.

In some embodiments, the method includes isolating (e.g., blocking via a valve) the test fluid in the metering chamber to prevent the test fluid from contacting the one or more reagents during the filling of the metering chamber.

In some embodiments, a second applied positive or negative pressure is applied by, or generated from, the measurement system (e.g., applied at a second pressure port in communication with the) at a second port in communication with the serpentine pathway so as to move the at least one aliquot in the second direction.

In some embodiments, the first applied positive or negative pressure is applied by, or generated from, the measurement system in reversed so as to move the at least one aliquot in the second direction.

In some embodiments, the operation of receiving the mixed aliquot in the testing chamber further comprises receiving a negative pressure via the third pressure port, wherein the third pressure port is further in fluid communication with the testing chamber.

In some embodiments, the testing chamber is downstream of the serpentine pathway and the third pressure port is downstream of the testing chamber.

These and other features and advantages of the present invention will become more readily apparent to those skilled in the art upon consideration of the following detailed description and accompanying drawings, which describe both the preferred and alternative embodiments of the present invention.

### BRIEF DESCRIPTION OF THE DRAWINGS

The accompanying drawings, which are incorporated in and constitute a part of this specification, illustrate embodiments and together with the description, serve to explain the principles of the methods and systems:

FIG. 1 shows a perspective view of an example biological sample input of a cartridge for use in a disposable system, in accordance with an illustrative embodiment.

FIG. 2 shows a side cross-sectional view of the example biological sample input of FIG. 1 with a casing, in accordance with an illustrative embodiment.

FIG. 3 shows a side cross-sectional view of the example biological sample input of FIG. 2 with a sample holding tube attached thereon, in accordance with an illustrative embodiment.

FIG. 4 shows a detailed view of the example biological sample input of FIG. 3, in accordance with an illustrative embodiment.

FIGS. 5A and 5B each shows the biological fluid pathways of four testing circuits (e.g., Hemostasis testing circuits) that are located on a sample preparation plane, in accordance with an illustrative embodiment. FIG. 5B further shows the cartridge body of FIG. 5A further coupled to a sample holding tube, in accordance with an illustrative embodiment.

FIGS. 6A and 6B show a front perspective view and a back perspective view of FIGS. 5A, 5B, and 8 with labels corresponding to the heating chamber filling.

FIGS. 6C and 6D show a front perspective view and a back perspective view of FIGS. 5A, 5B, and 8 with labels corresponding to the sample chamber filling.

FIGS. 6E and 6F show a front perspective view and a back perspective view of FIGS. 5A, 5B, and 8 with labels corresponding to the sample mixing and test chamber filling.

FIG. 7 shows the back-side of the cartridge of FIG. 5A and includes an interconnection plane that interfaces to the sample preparation plane, that collectively form the biological fluid pathways for the testing circuits, in accordance with an illustrative embodiment.

FIG. 8 shows portions of the biological fluid pathways that are on the interconnection plane of FIG. 7, in accordance with an illustrative embodiment.

FIG. 9 shows an example testing chamber section for use with the example cartridge, in accordance with an illustrative embodiment.

FIG. 10 shows a cross-sectional view of an example testing chamber in the example testing chamber section, in accordance with an illustrative embodiment.

FIG. 11 shows a detailed cross-sectional view of the example testing chamber of FIG. 10, in accordance with an illustrative embodiment.

FIG. 12 shows a cross-sectional view of the disposable system operatively coupled to a measurement system, in accordance with an illustrative embodiment.

FIG. 13 shows an example shear modulus versus time curve, in accordance with an illustrative embodiment.

FIG. 14 shows an example of shear modulus curves obtained with an activator of coagulation and with and without a fibrinolysis inhibitor. A differential comparison of these curves can provide information about the fibrinolytic activity of the sample.

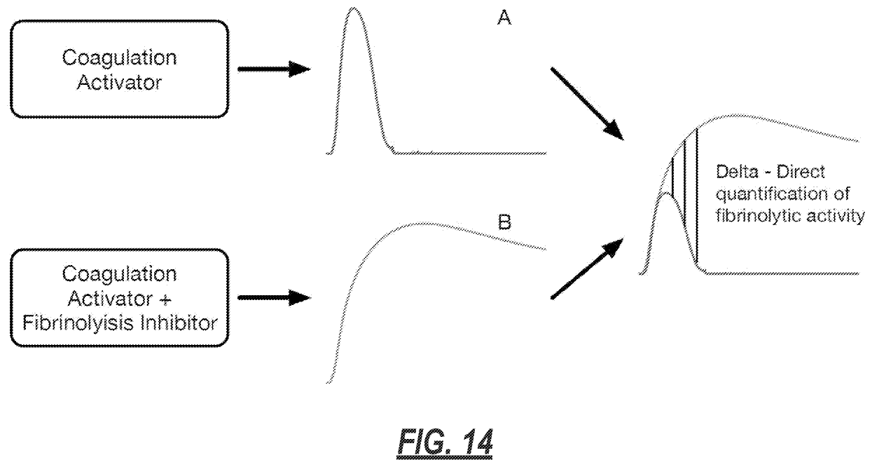

FIG. 15 shows potential embodiments of differential metrics that can be measured from shear modulus curves obtained with an activator of coagulation and with and without a fibrinolysis inhibitor.

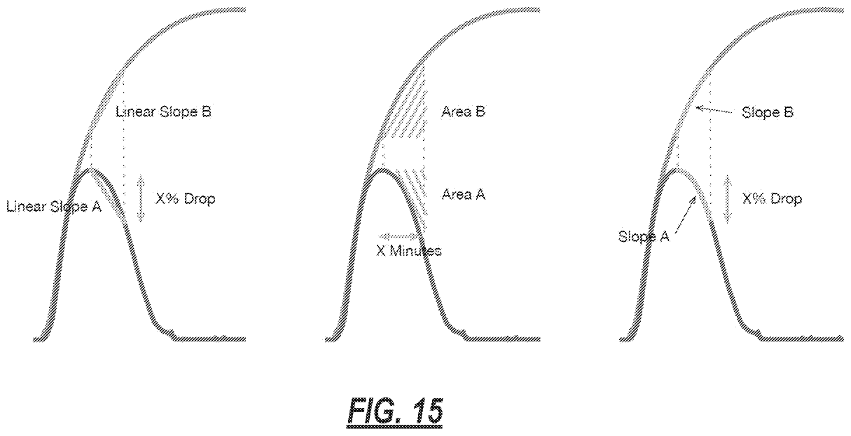

FIG. 16 shows a photograph of an exemplary cartridge of FIGS. 1, 2, 3, 4, 5A, 5B, 7, 8, and 9 for use in a disposable system, in accordance with an illustrative embodiment.

FIG. 17 shows a front view of the exemplary cartridge of FIGS. 1, 2, 3, 4, 5A, 5B, 7, 8, and 9 in accordance with an illustrative embodiment.

FIG. 18 shows a front view of the exemplary cartridge of FIGS. 1, 2, 3, 4, 5A, 5B, 7, 8, and 9, in accordance with an illustrative embodiments.

### DETAILED DESCRIPTION

The present invention now will be described more fully hereinafter with reference to specific embodiments of the invention. Indeed, the invention can be embodied in many different forms and should not be construed as limited to the embodiments set forth herein; rather, these embodiments are provided so that this disclosure will satisfy applicable legal requirements.

As used in the specification, and in the appended claims, the singular forms “a,” “an,” “the,” include plural referents unless the context clearly dictates otherwise.

The term “comprising” and variations thereof as used herein are used synonymously with the term “including” and variations thereof and are open, non-limiting terms.

As used throughout, by a “subject” is meant an individual. The subject may be a vertebrate, more specifically a mammal (e.g., a human, horse, pig, rabbit, dog, sheep, goat, non-human primate, cow, cat, guinea pig or rodent), a fish, a bird or a reptile or an amphibian. The term does not denote a particular age or sex.

The apparatus described here includes a single-use cartridge apparatus configured to facilitate in vitro assessment of one or more hemostatic functions. Hemostatic function refers to a functional role of various blood components such coagulation factors, fibrinogen, platelets, fibrinolytic factors, and components of the vasculature. The cartridge apparatus and associated measurement system, in some embodiments, are configured to assess hemostatic function by measuring changes in at least one mechanical property of the tested sample when such sample is exposed to one or more reagents. In some embodiments, the cartridge apparatus and its test chambers are configured to facilitate measurements of viscoelastic properties, e.g., based on interrogation using ultrasound pulses or ultrasonic energy. However, other interrogation systems may be used with a cartridge apparatus with the features described herein. Examples of other interrogation systems includes, for example, but not limited to, systems that employ cup/pin technologies (such as in the case of thromboelastography and thromboelastometry), oscillating piston to measure changes in mechanical impedance, optical sensing, fluorescence sensing, colorimetric sensing, aggregometry, resonance sensing, or electrical impedance sensing, among others.

A broad array of reagents can be utilized in the cartridge apparatus, including intrinsic pathway activators (without limitations kaolin, Hageman factor, celite, glass, ellagic acid, micronized silica etc), extrinsic pathway activators (without limitations tissue factor, recombinant tissue factor, thromboplastin, etc), other coagulation activators (without limitations thrombin, factor Xa, reptilase, ecarin, Russell's viper venom or other snake venoms, etc), platelet activators or platelet inhibitors (without limitations GPIIb/IIIa inhibitors (such as abciximab, eptifibatide, tirofiban, roxifiban, orbofiban), cytochalasin D, blebbistatin, AR1 inhibitors, PAR4 inhibitors, glycoprotein IB inhibitors, TRAP, ADP, arachidonic acid, ADP inhibitors, non-steroidal anti-inflammatory drugs, etc.), fibrinolytic function activators or fibrinolytic function inhibitors (without limitations tPA, uKA, streptokinase, TAFIa, plasmin/plasminogen, aprotinin, epsilon-aminocaproic acid, tranexamic acid, plasminogen activator inhibitor 1 (PAI1), αa2-antiplasmin (α2-AP), or plasmin-antiplasmin complexes, carboxypeptidase inhibitor, etc.), and others (FXIIIa inhibitors, Hexadimethrine bromide (polybrene), heparinase (e.g., heparinase I), ristocetin, heparin, low molecular weight heparin, corn trypsin inhibitor, adenosine, GPRP, calcium, fibronectin, collagen, epinephrine, immuno-detection reagents, direct thrombin inhibitors, factor Xa inhibitors, reagents aimed at reversing or eliminating the effects of the new oral anticoagulants (such as the direct thrombin inhibitors and the factor Xa inhibitors), thrombomodulin, etc.). Additional non-functional reagents could also be used to preserve the functionality of the other reagents (buffers and stabilizers for lyophilization or drying, dyes, etc.).

Reagents, in some embodiments, are placed and stored in chambers (e.g., pockets located within a fluidic circuit) in the cartridge apparatus but in alternative embodiments reagents can be placed and stored in various chambers or fluidic channels in the fluidic circuit of the cartridge apparatus. A fluidic circuit generally refers to one or more fluidic pathways established between sample preparation and the one or more test chambers where samples are ultimately measured.

In some embodiments, reagents are placed and stored in the cartridge apparatus in liquid forms or can be lyophilized in spheres (such as in the case of the Lyopheres™ produced by BioLyph LLC), lyophilized in films, lyophilized on the plastic surfaces, dried on the plastic surfaces, or spray coated, etc., in order to improve shelf-life stability. A person of ordinary skills in the art should recognize that these reagents are not fully inclusive and other reagents or reagent combinations that are inhibitors or activators of one or more hemostatic functions could be used in this cartridge.

The cartridge apparatus disclosed here is a component of a measurement system (e.g., a hemostasis measurement system). The measurement system (also referred to as the instrument) includes at least an interface element which couples between the cartridge apparatus and a measuring element configured to measures viscoelastic properties or mechanical properties of a sample processed within the cartridge apparatus. The measured viscoelastic properties or mechanical properties are outputted as results to a user interface. An example user interface is described in commonly assigned U.S. Pub. No. 2011/0252352 to Viola et al., which is incorporated by reference herein in its entirety.

In some embodiments, the interface element includes one or more heating and/or cooling elements.

In some embodiments, the interface element includes a fluidic manifold that facilitate connection to one or more pump elements and one or more valves.

In some embodiments, the interface element includes one or more sensors, e.g., configured to perform hemostasis measurements. The one or more sensors, in some embodiments, includes ultrasound sensors. In other embodiments, the one or more sensors includes other interrogative devices that is based on thromboelastography, thromboelastometry (e.g., a thromboelastography-based system or a thromboelastometry-based system), or that measures changes in mechanical impedance, changes in perturbation as observed via an optical-based system (e.g., having an optical sensor), fluorescence, colorimetric-based system, aggregometry-based system (e.g., having optical sensor, acoustic sensor, or electrodes that measure aggregation with the test sample), resonance-based system (e.g., having optical, acoustic, or mechanical position sensors that measures the sample when the sample is at, or near resonance), electrical impedance-based system (e.g., having electrodes configured to measure electrical impedance), or a combination thereof.

In some embodiments, the interface element includes a mechanical clamp configured to position the cartridge apparatus in a desired orientation with respect to the components (the one or more sensors, the fluidic manifold, the heating and/or cooling elements, and etc.) of the measurement system. When the interface element is interfaced with the components of the measurement system, the cartridge apparatus, in some embodiments, is driven via a series of controlled actions orchestrated by the measurement system to prepare the test sample for measurement. In some embodiments, the preparation operations include sample aspiration of a sample from a sample container (also referred to as a sample holding tube), sample heating and/or cooling, sample metering, sample mixing with reagents, and sample measurement. Each step, with reference to various embodiments, is described below. After measurements are completed, the results are output in the instrument user interface.

In some embodiments, the cartridge apparatus and its internal components are the only component that directly contact with a sample to be analyzed.

In some embodiments, the cartridge includes computer readable information that can be optically or communicatively interrogated (e.g., RFID tags, computer readable medium such as flash ICs, QR codes, BAR codes, and etc.) and/or human readable information (e.g., labels).

The various embodiments described below does not utilize any active valve element in the cartridge design, but instead relies on a fluidic manifold and one or more valves placed in the instrument. Fluid is moved through the various cartridge components via pressure differential and/or gravity and/or material properties (such as in the case of hydrophobicity or hydrophilicity) and/or capillary forces.

In these embodiments, the cartridge is configured to couple with the instrument via one or more connection ports that are aligned via alignment slots. The connection ports include one or more pressure ports and one or more vent ports. However, in alternative embodiments, actuated valves (such as in the case of elastomeric valves) can be included in the cartridge design to control fluid flow. These valves are actuated, in some embodiments, by corresponding hardware and software components in the measurement system.

The surface properties and texture of the cartridge surfaces in direct contact with the sample can be optimized to promote sample adhesion and/or sample flow. In some embodiments, the test chamber's interior surface and/or other interior surfaces of the fluidic circuit within the cartridge apparatus are plasma treated to optimize the surface energy and texture for adhesion of specific plasma proteins. In other embodiments, test chamber's interior surface and/or other interior surfaces of the fluidic circuit are treated with surface roughness texturing, material coating (such as in the case of gold plating), biological material coating (such as in the case of fibronectin or collagen coating, for example), raw material selection (e.g., use of specific plastic or other materials for the plate that does not require additional treatment), etc. Such treatments maybe performed independently, or in conjunction with, a plasma treatment. Similarly, the cartridge materials can be selected or manipulated to achieve the desired ydrophobicity or hydrophilicity. These properties can be changed by plasma treatment or by surface coatings.

As described in more details below, the cartridge and the associated measurement system can utilize one or more sensors of one or more types (e.g., optics, pressure, ultrasound, etc.) as part of the automated operations of the cartridge. In addition, the outputs of such one or more sensor(s) can be further utilized to perform quality control checks. These checks may be performed before, during, or after cartridge testing to ensure function of one or more of the subsystems (for example, ultrasound or other interrogation system, fluidics, fluid level, clamping, cartridge positioning/orientation system, or temperature control), ensure the cartridge is functional, ensure correct sample preparation before measurements are performed or have been performed for the measurement, and may also be used to accept or reject a test result or even to abort testing before initiation of measurements.

Note that in the discussion below a fluidic circuit includes a channel with fluidic component that connects one or more chambers together. Fluid circuit is also referred to as a testing channel in a multitude of channels that can be individually and controllably processed within a single cartridge apparatus.

### Cartridge Input Section

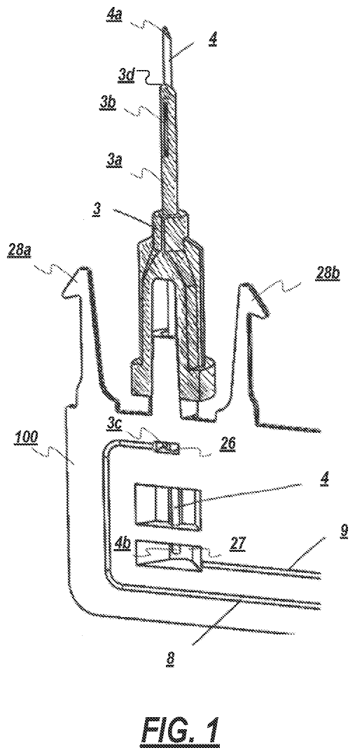

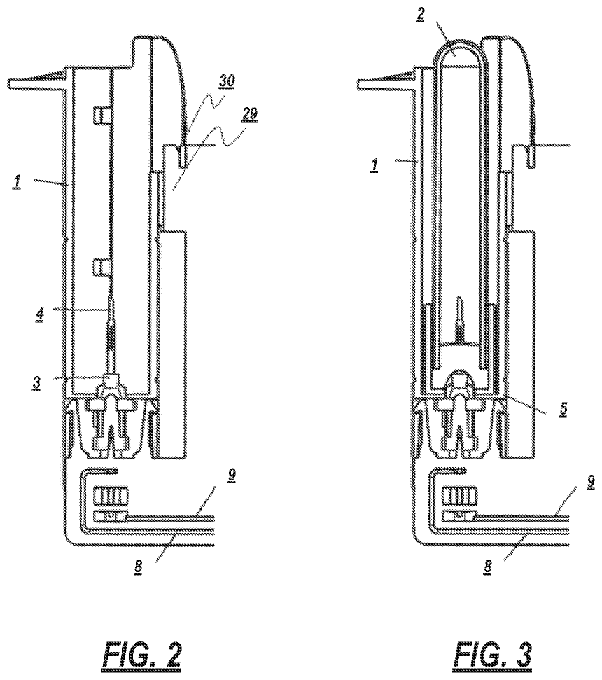

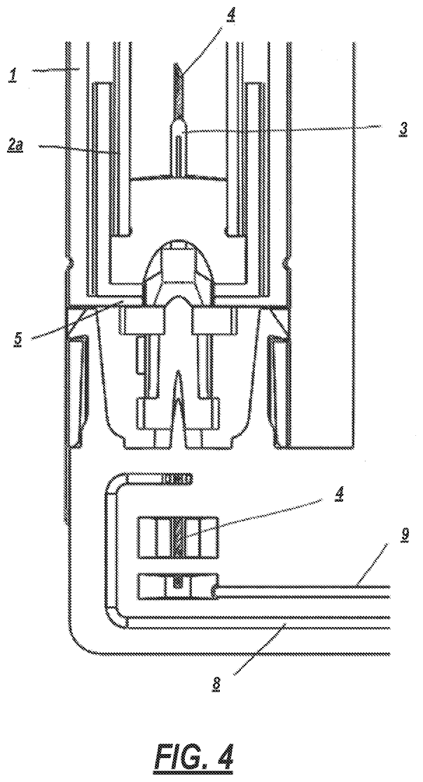

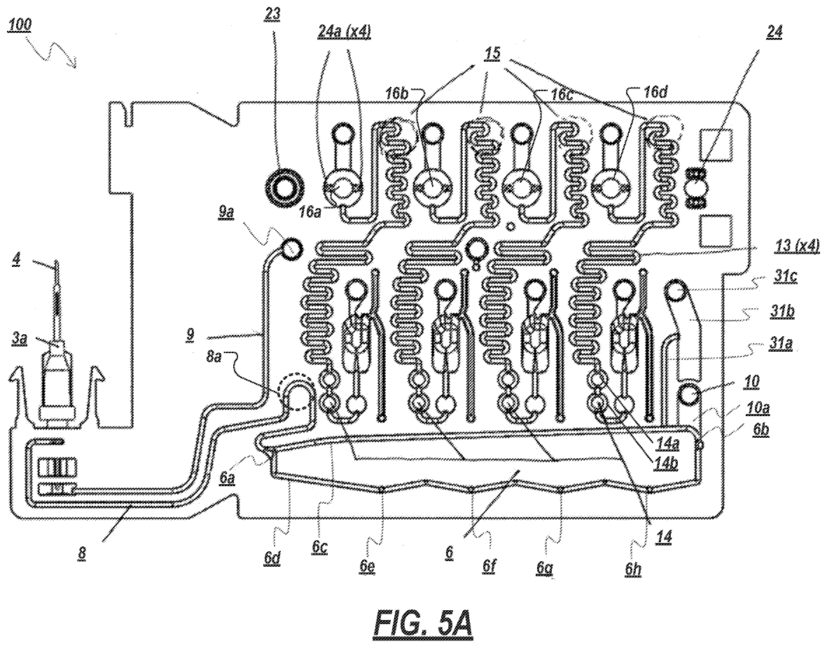

FIGS. 1, 2, 3, and 4 are schematic illustrations of an example biological sample input section of a cartridge 100 for evaluating hemostasis. Specifically, FIG. 1 shows a perspective view of an example biological sample input of a cartridge for use in disposable system, in accordance with an illustrative embodiment. FIG. 2 shows a side view of the example biological sample input of FIG. 1 with a casing, in accordance with an illustrative embodiment. FIG. 3 shows a side cross-sectional view of the example biological sample input of FIG. 2 with a sample holding tube attached thereon, in accordance with an illustrative embodiment. FIG. 4 shows a detailed view of the example biological sample input of FIG. 3, in accordance with an illustrative embodiment. In alternative embodiments, the input section of the cartridge comprises a well to which fluid sample can be placed, for example, by way of a pipette or tube.

In some embodiments, and as shown in FIG. 1, the cartridge 100 has a dual connection tab 28a, 28b for coupling the cartridge 100 to a sample container guide 1 (shown in FIG. 2). As shown in FIG. 2, the sample container guide 1, when mated with the cartridge 100, aligns a sample container 2 to a sample input port 3 of the cartridge 100. The cartridge 100 also includes an alignment tab 29 that is configured to slide into an alignment groove 30 of the sample container guide 1 to further stabilize the coupling of the sample container guide 1 to the cartridge 100. The sample container guide 1 can further provide a hard stop 5 (shown in FIG. 3) to hold the sample container 2 at the appropriate height to establish fluidic communication with the cartridge 100.

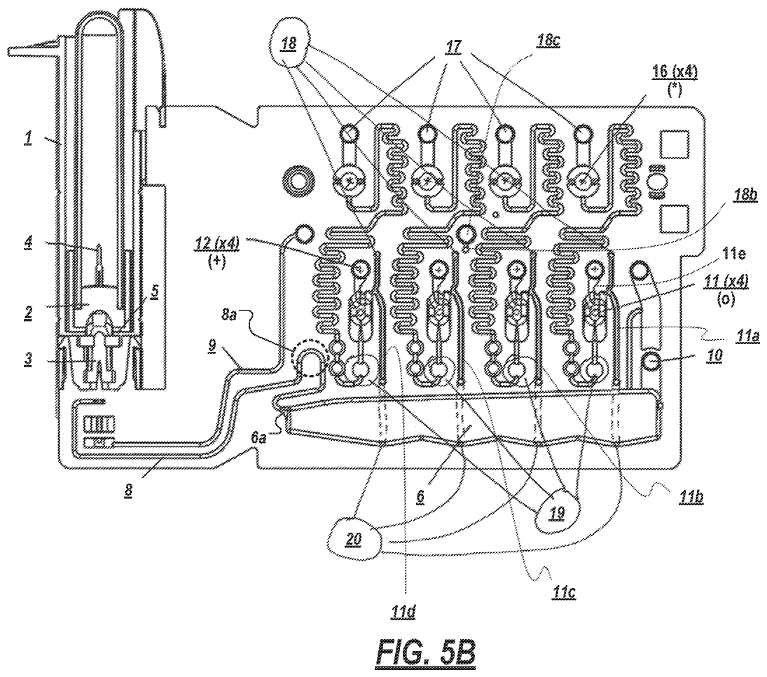

In various embodiments, the sample container 2 is an evacuated tube (also referred to herein as the sample holding tube 2) such as a BD Vacutainer™ tube, and the sample input port 3 comprises one or more needles required for sample transferring 3a and venting 4 (see FIG. 1). Though shown as concentric in the figures, the needles can be configured to be concentric, side by side, or integrated. In some embodiments, and as shown in FIG. 1, the sample transferring needle 3a includes inlets (3b and 3d) and an outlet 3c that terminates in a sample inlet chamber 26 of the cartridge 100. The sample inlet chamber 26 is in fluid communication with an inlet pathway 8 that leads to a retention/heating chamber 6 (see FIG. 5A). In some embodiments, and as shown in FIG. 1, the venting needle 4 includes an outlet 4a that is configured to terminate within the sample container 2 when attached and is spaced apart from the inlet 3d so as to minimize bubbles being drawn into the inlets 3b and 3d. The venting needle 4 also has an inlet 4b that terminates in a venting inlet chamber 27 of the cartridge. The venting inlet chamber 27 is in fluid communication with a vent pathway 9 that, in some embodiments, terminates at a filter chamber 9a (shown in FIG. 5A) housing a filter. An alternative sample container 2 can be utilized, such as a syringe, which requires a luer lock connection on the cartridge 100. And, as noted above, in other embodiments, the input section of the cartridge comprises a well to which fluid sample can be placed, for example, by way of a pipette or tube.

### Vent Pathway

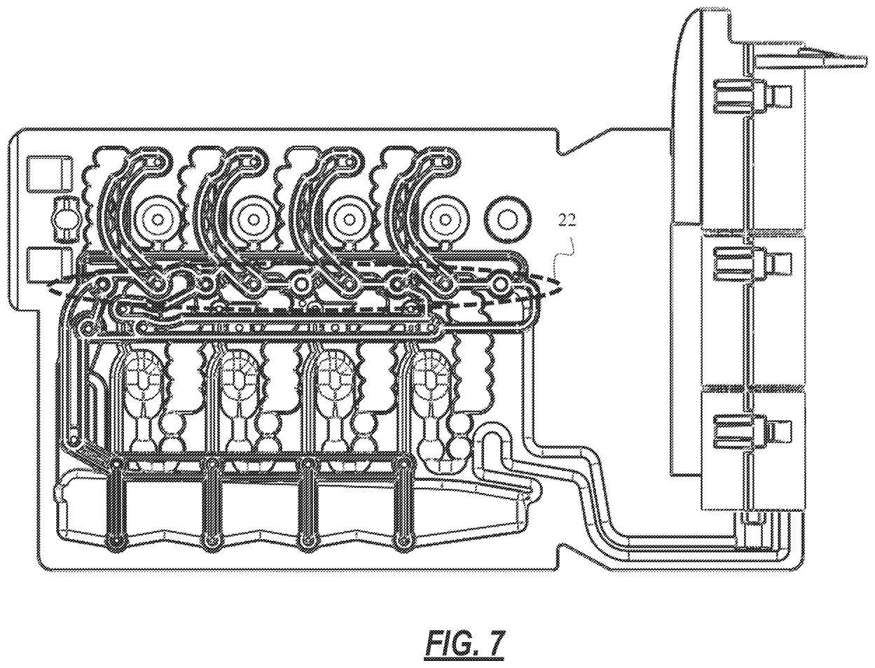

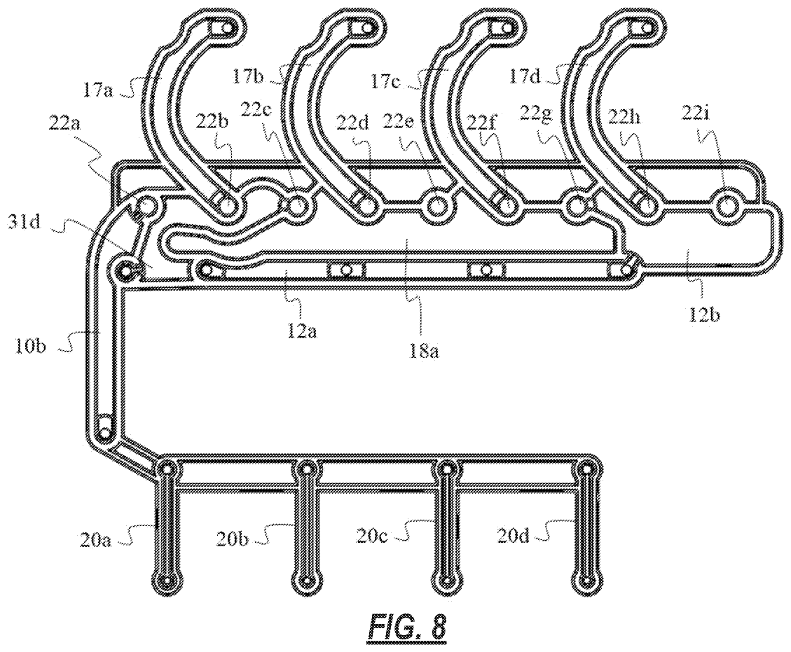

FIGS. 5A, 5B, 7, and 8 are schematic illustrations of biological fluid pathways of the example cartridge 100 in accordance to an embodiment. Specifically, FIGS. 5A and 5B each shows the biological fluid pathways of four testing circuits (corresponding to test chambers 16a, 16b, 16c, and 16d, shown in FIG. 5A), also referred to herein as Hemostasis testing circuits, that are located on a sample preparation plane, in accordance with an illustrative embodiment. Though shown with four testing circuits, additional circuits or less may be included, including, e.g., two, three, five, six, seven, eight, and etc. FIG. 5B further shows of the cartridge body of FIG. 5A further coupled to a sample holding tube 2, in accordance with an illustrative embodiment. FIG. 7 shows the back-side of the cartridge of FIG. 5A and includes an interconnection plane that interfaces to the sample preparation plane, that collectively form the biological fluid pathways for the testing circuits, in accordance with an illustrative embodiment. FIG. 8 shows portions of the biological fluid pathways that are on the interconnection plane of FIG. 7, in accordance with an illustrative embodiment.

As noted above, the biological fluid pathways are formed on, and across, multiple planes defined in the cartridge 100. A first plane of fluid pathways of the cartridge 100 is shown in FIGS. 5A and 5B. The first plane of fluid pathways of the cartridge 100 may alternatively be referred to as a front plane of the cartridge 100. FIGS. 7 and 8 each shows a second plane of fluid pathways of the cartridge 100 in which FIG. 8 shows the fluid pathways in the second plane isolated from the remainder of the structure of the cartridge 100 for ease of understanding. The second plane of fluid pathways of the cartridge 100 may alternatively be referred to as a back plane of the cartridge 100. The fluid pathways between the first and second planes are connected by fluidic vias that traverse across the various planes of the cartridge 100.

As discussed above in relation to FIG. 1, in some embodiments, the venting inlet chamber 27 is in fluid communication with a vent pathway 9. The vent pathway 9 may terminate at a filter chamber 9a (shown in FIG. 5A), which may house a filter therein. The filter chamber 9a in the first plane of fluid pathways of the cartridge 100 is in fluid communication with a vent port 22i (shown in FIG. 8) in the second plane of fluid pathways of the cartridge 100. As discussed in more detail below, cartridge 100 couples with the measurement system (also referred to herein as the instrument) via the vent port 22i to provide atmospheric pressure to vent pathway 9.

### Heating Chamber Pathway

As discussed above in relation to FIG. 1, in some embodiments, the sample transferring needle outlet 3c terminates in a sample inlet chamber 26 of the cartridge 100. The sample inlet chamber 26 is in fluid communication with an inlet pathway 8. The sample inlet pathway 8 provides a fluid communication pathway between the sample inlet chamber 26 and a retaining/heating chamber 6 (also referred to herein as heating chamber 6 or as a “first chamber”) (shown in FIG. 5A). The label “first”, “second”, and “third” as used herein is provided merely as labels and do not intended to connote a sequence. The heating chamber 6 is configured to mate with a corresponding thermal regulating (e.g., heating/cooling) system in the measurement system to warm or cool the sample toward or to, or near, a pre-defined temperature.

The heating chamber 6, as provided herein, facilitate uniform conditioning of the test fluid prior to the fluid be metered or aliquoted to their respective testing, thus reducing variability in the test sample that can affect subsequent measurements and analysis. The shape of the heating chamber 6 can be optimized for heating/cooling transfer, as in the case here in which a thin cross-section with thin walls is used. The materials of the cartridge 100 can also be optimized to facilitate heating/cooling. In some embodiments, the sample heating/cooling conditioning stage can also be implemented in one or more chamber/channels of the cartridge design and it is not limited to just occur within just the heating chamber 6. In some embodiments, a stirring, rotating, or oscillating element (not shown) can be placed in the heating chamber 6 that may be controlled by the measurement system to promote uniform temperature heating or cooling. In other embodiments, test fluid in the heating chamber 6 may be vibrated by the measurement system vibrating the cartridge 100 to promote uniform temperature conditioning of the test fluid.

In some embodiments, temperature measurement is conducted of the test sample in the cartridge 100. To measure the temperature, a sensor can be incorporated in the measurement system or in the cartridge 100. In some embodiments, a thermistor or thermocouple can be placed in physical contact with the cartridge 100, or biological sample (such as blood). In other embodiments, an IR thermometer is pointed at the cartridge 100 or biological sample. In either case the cartridge 100 may incorporate a small well through which the incoming blood passes, rather than having direct contact with the blood. In some embodiments, the temperature of the test sample may be assessed at or near the heating chamber 6. In other embodiments, the temperature of the test sample may be assessed while the test sample is flowing through channels as it is directed toward the test chambers 16.

Referring now to FIGS. 5A, 5B, and 8, the sample inlet pathway 8 terminates at a first corner 6a of the heating chamber 6, shown as the top left corner of heating chamber 6 in FIGS. 5A and 5B. In some embodiments, chambers along the fluid pathways are generally filled from the top as to prevent blood to backflow into the inlet. A fill outlet channel 10a extends from a second corner 6b of the heating chamber 6 opposite from the first corner 6a.

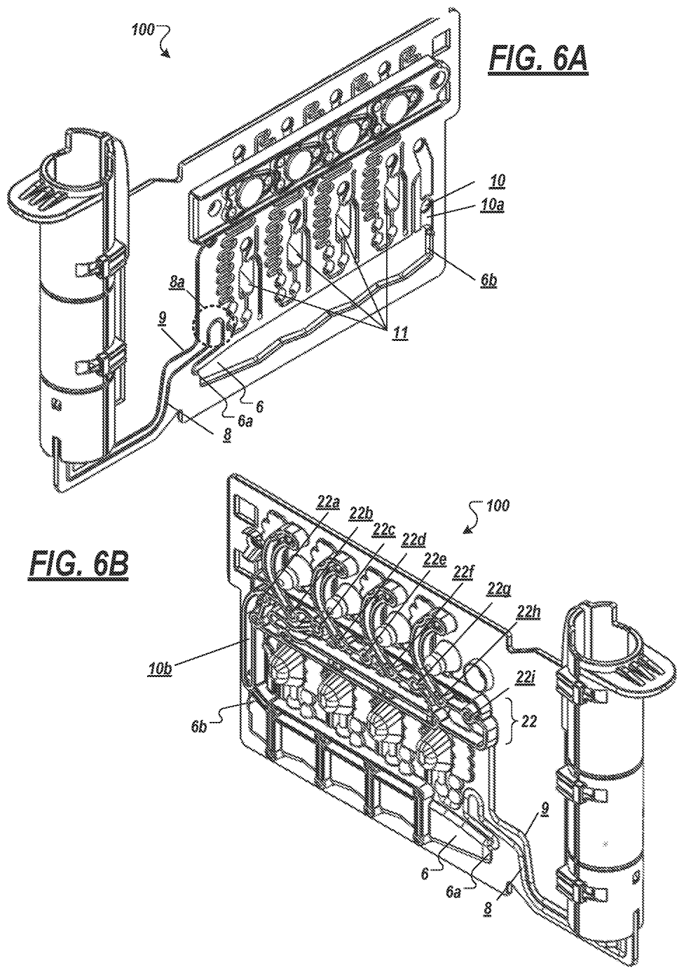

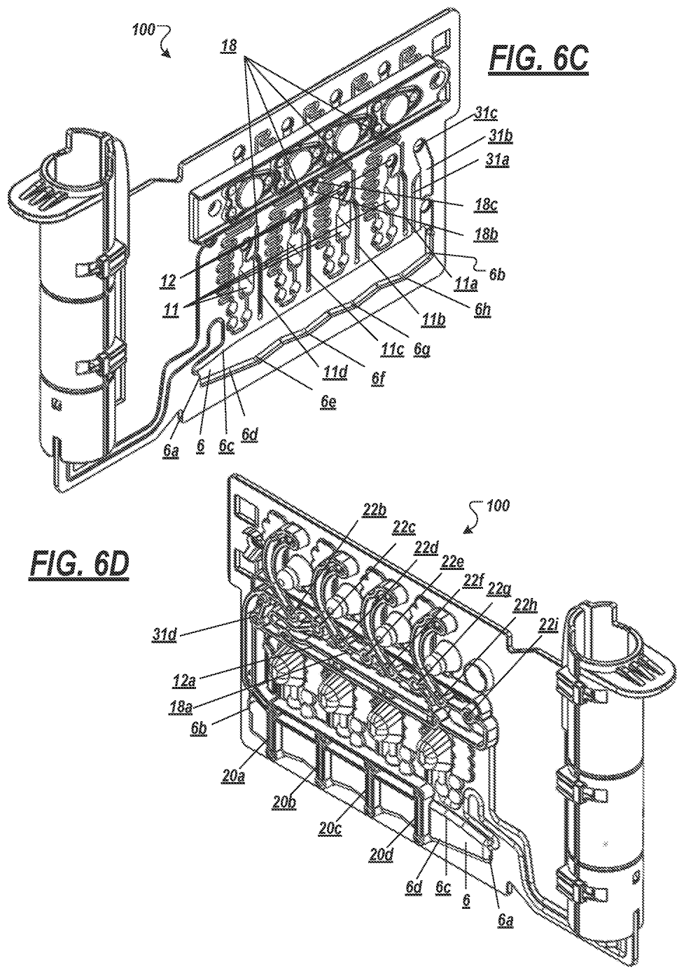

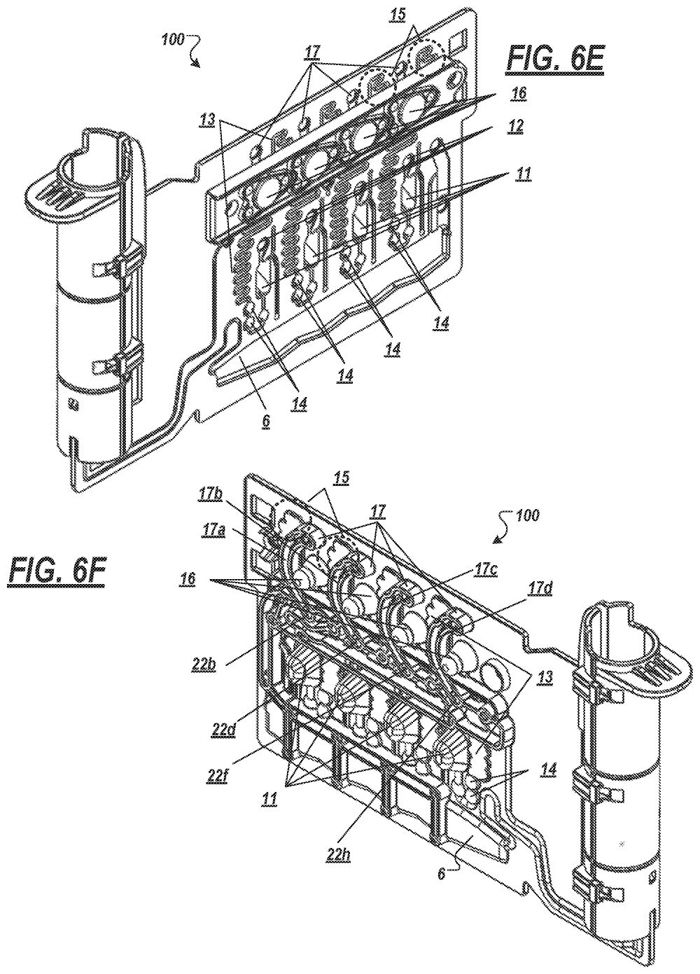

The fill outlet channel 10a extends to a filter chamber 10 with a filter therein. The filter chamber 10 (e.g., as shown in FIGS. 5A and 5B) in the first plane of fluid pathways of the cartridge 100 is in fluid communication with a heating-chamber fill channel 10b shown in the second plane of fluid pathways of the cartridge 100 (see FIG. 8). The fill conduit 10b is in fluid communication with a pressure port 22a (see also FIG. 8) that facilitate filling of the heating chamber 6. The conduit 10b is a part of a network of conduits used to consolidate pressure ports (e.g., 22a as discussed, as well as 22b-22i to be later discussed) of the cartridge 100 to one or more areas to which the measurement system can coupled with its pressure control interfaces. Such configuration reduces the complexity of the measurement system to control fluid movement within the cartridge 100. Indeed, the conduits that handles the control of movement of the fluid sample in the first plane of the cartridge 100 is primarily placed in the second plane of the cartridge 100. FIGS. 6A and 6B show a front perspective view and a back perspective view of FIGS. 5A, 5B, and 8 with additional labels corresponding to the description of this section.

### Heating Chamber Fill

In operation, the instrument's fluid pump aspirates the sample through the input port 3 (see FIGS. 1-2) via the connection ports 22 (see FIG. 7) (also referred to herein as pressure ports) and into the heating chamber 6 (see FIG. 5A or 5B) of the cartridge 100. For example, the instrument's fluid pump may be in communication with and apply differential pressure (e.g., positive or negative) to a pressure port 22a (see FIG. 8). This in turn creates an applied pressure along the fill conduit 10b, within the heating chamber 6, and along the inlet pathway 8 to aspirate the sample into the heating chamber 6. At the same time, the inner vent needle 4 is linked to the isolated pathway 9 that receives atmospheric pressure from the instrument via the vent port 22i (see FIG. 8) to neutralize pressure in the sample container 2 as the sample is aspirated into the heating chamber 6 of the cartridge 100. During filling of the heating chamber 6, all other ports (e.g., 22b-22i) are closed, e.g., by the measurement system.

When the heating chamber 6 is filled, the filter within filter chamber 10 is clogged and creates a pressure spike that is detected by the instrument, causing the instrument to turn off the fluidic pump. The instrument may also close the vent port 22i or otherwise discontinue supplying atmospheric pressure via vent port 22i upon detecting the pressure spike. Alternative filling detection techniques could also be used, i.e., optical sensors placed at the desired fill level, volumetric control, fixed time of pressure alteration (negative and/or positive pressures), ultrasound detectors placed at the desired fill level, etc. The sample remains in the heating chamber until the desired temperature is reached, which can for example be at or near body temperature of a normal and typical subject (e.g., about 37° C. for a healthy person). In other instances, other desired temperatures may be warranted. The shape of the heating chamber 6 and the channels leading to the sample metering chambers 11 (described below) are configured so that bubbles that might be present in the fluid sample are trapped away from the rest of the fluidic circuit. The shape of the inlet pathway 8 includes an anti-siphon feature 8a (see FIGS. 5A and 5B) and is configured to reduce the occurrence of bubbles forming in the heating chamber 6 and prevent siphoning to and from the sample container 2. To this end, additional unprocessed test sample (e.g., un-warmed blood) is prevented from being siphoned into the heating chamber following the first drawn test sample being heated and/or cooled, e.g., when the processed test sample is pulled into the metering chamber 11 (also referred to herein as sample chamber 11 and “second” chamber). FIGS. 6A and 6B also show labels corresponding to the description of this section.

### Sample Aliquot (Metering) Chambers Pathway

Referring to FIGS. 5A, 5B, and 8, a first side 6c of the heating chamber 6 (see FIG. 5A) extends between the first corner 6a and the second corner 6b.

One or more of outlet ports 6e-6h (see FIG. 5A) are arranged along the length of the second side 6d of the heating chamber. Each of the one or more outlet ports 6e-6h may be arranged in a different one of the valleys along the second side 6d of the heating chamber in which the valleys direct the sample into conduits leading to each respective test channel. A test channel, in some embodiments, refers to the associated fluidic pathway structures and testing chamber, collectively, used to perform a measurement for a given aliquot sample. In some embodiments, and as shown in FIG. 5B, each of the one or more outlet ports 6e-6h in the first plane of fluid pathways of the cartridge 100 is in fluid communication with a first end of a corresponding one or more channels 20a-20d (see FIG. 8) in the second plane of fluid pathways of the cartridge 100, collectively referred to as channels 20 (see FIG. 5B). A second end of each of the one or more channels 20a-20d (see FIG. 8) in the second plane of fluid pathways of the cartridge 100 is likewise in fluid communication with a first end of a corresponding one or more channels 11a-11d (see FIG. 5B) in in the first plane of fluid pathways of the cartridge 100. A second end of each of the one or more channels 11a-11d terminates in a corresponding one or more sample chambers 11 (shown in duplicates (“×4”) in FIG. 5B with an “o” symbol therein). In the example shown in FIGS. 5A, 5B, 7, and 8, there are four sample chambers 11. More or fewer sample chambers 11 and corresponding fluid communication pathways with the heating chamber 6 may be present on the cartridge 100 in some configurations.

The sample chambers 11 are fed by the one or more channels 20 originating from the bottom of the heating chamber 6. This geometric configuration avoids bubbles being drawn into the sample chambers 11 as the bubbles rise to the upper portion of the heating chamber 6.

Each of the sample chambers 11 has a corresponding fill channel 11e that is in fluid communication with a corresponding filter chamber 12 (shown in duplicates (“×4”) in FIG. 5B with a “+” symbol) with a filter therein. The filter chamber 12 in the first plane of fluid pathways of the cartridge 100 is in fluid communication with a channel 12a (see FIG. 8) in the second plane of fluid pathways of the cartridge 100. The channel 12a is in fluid communication with a pressure port 22g (see FIG. 8).

In some configurations, when more than one sample chamber 11 are implemented on the cartridge 100, the channel 12a (see FIG. 8) is in fluid communication with all of the sample chambers 11 (see FIG. 5B) via corresponding fill channels 11e and filter chambers 12. Accordingly, channel 12a acts as a manifold for applying negative pressure to all of the sample chambers 11 via a single pressure port 22g. Therefore, a separate pressure port is beneficially not needed for filling each of the sample chambers 11. FIGS. 6C and 6D show a front perspective view and a back perspective view of FIGS. 5A, 5B, and 8 with additional labels corresponding to the description of this section.

### Heating Chamber Vent Pathway

Referring to FIGS. 5A, 5B, and 8, the heating chamber 6 comprises a vent pathway 31a, 31b, 31c along the first side 6c of the heating chamber for venting the heating chamber 6 as the sample chambers 11 are filled. The vent pathway 31 (not shown) includes the fluid pathway through conduit elements 31a-31d. Channels 31a-31b terminate at one end in the heating chamber 6 along the first side 6c and terminate at the other end at a filter chamber 31c with a filter therein. Accordingly, channels 31a-b provide a fluid pathway between the heating chamber 6 and the filter chamber 31c. The filter chamber 31c (see FIG. 5A) in the first plane of fluid pathways of the cartridge 100 is in fluid communication with a channel 31d (see FIG. 8) in the second plane of fluid pathways of the cartridge 100, which in turn is in fluid communication with a vent port 22c (see FIG. 8). As discussed in more detail below, cartridge 100 couples with the instrument via the vent port 22c for the instrument to provide atmospheric pressure to vent the heating chamber 6. FIGS. 6C and 6D also show labels corresponding to the description of this section.

### Sample Chamber Vent Pathway

Referring to FIGS. 5A, 5B, and 8 each of the sample chambers 11 includes a vent pathway 18 that terminates at a first end in a corresponding sample chamber 11. A second end of the vent pathway 18 in the first plane of fluid pathways of the cartridge 100 is in fluid communication with a vent manifold 18 in the second plan of fluid pathways of the cartridge 100. When more than one sample chamber 11 is present, all of the vent pathways 18 (see FIG. 5B) of the sample chambers are in fluid communication with the vent manifold 18a (see FIG. 8). The vent manifold 18a (see FIG. 8) in the second plane of fluid pathways of the cartridge 100 is further in fluid communication with a first end of a channel 18b (see FIG. 5B) in the first plane of fluid pathways. A second end of channel 18b (see FIG. 5B) is in fluid communication with a filter chamber 18c (see FIG. 5B) with a filter therein. Filter chamber 18c (see FIG. 5B) in the first plane of fluid pathways of the cartridge 100 is in fluid communication with a vent port 22e (see FIG. 8). FIGS. 6C and 6D also show labels corresponding to the description of this section.

### Sample Aliquot (Metering) Chambers Fill

During operation, once the sample is at, or near, the desired temperature, the sample is aliquoted (or metered) into one or more independent sample chambers 11 (see FIG. 5B). Referring to FIG. 5B unless indicated otherwise, in various embodiments, the sample chambers are filled by applying a negative pressure at pressure port 22g (see FIG. 8) via a pump in the instrument while venting the heating chamber 6 by way of the vent port 22c (see FIG. 8). Each filter chamber 12 has a filter therein that will clog when the corresponding sample chamber 11 is filled and will trigger the pressure sensor of the instrument to turn the pump off, similar to filling the heating chamber 6. As discussed above, alternative filling detection techniques could be used. In various embodiments, all the sample chambers 11 are controlled by a single valve and fluidic pathway in the instrument via pressure port 22g (see FIG. 8). The cutoff pressure will not trigger until all sample chambers' filters clog in the corresponding filter chambers 12. The sample chambers 11 are used to separate samples into independent functional channels, aliquot a known volume of sample, and stage the sample for mixing with reagents. While the sample chambers 11 are filled, pressure ports 22b, 22d, 22f, and 22h (see FIG. 8) as well as vent port 22e (see FIG. 8) are closed by the instrument so as to prevent fluid from leaking past the location 19, arranged below the sample chambers 11. Once the sample chambers 11 have been filled, the vent port 22e (see FIG. 8) is opened to atmospheric pressure so that the one or more sample chambers 11 can be fluidically separated from each other and the heating chamber 6. Vent port 22e (see FIG. 8) remains open to atmosphere during sample mixing as discussed below. FIGS. 6C and 6D also show labels corresponding to the description of this section.

### Mixing and Testing Pathway

Referring to FIG. 5B unless indicated otherwise, each of the sample chambers 11 is in fluid communication with a corresponding one or more reagent pocket 14 (see FIG. 5A) configured to house at least one lyophilized bead comprising a reagent. As shown in FIGS. 5A and 5B, two reagent pockets 14 (shown as 14a and 14b in FIG. 5A for one of the test channels) are provided. In other embodiments, a single reagent pocket is used for each testing channel. In yet other embodiments, more than two reagent pockets 14 are used for each testing channel. The reagent pockets 14 are in turn in fluid communication with a serpentine channel 13 (see FIG. 5A, and shown with a duplication symbol (“×4”). Each of the serpentine channel 13 has a first end in fluid communication with the reagent pockets and a second end that terminates at an optical detection zone 15. As discussed below, the instrument (i.e., measurement system) may optically interrogate the optical detection zone 15 of the cartridge 100 to facilitate the control of a pump that facilitates mixing of the individual aliquots with the corresponding reagent(s). Each of the serpentine channel 13 (see FIG. 5A) is in fluid communication with a test chamber 16 (see FIG. 5B, with duplication symbol “×4”) which in turn is in fluid communication with a filter chamber 17, with a filter therein. The filter chamber 17 in the first plane of fluid pathways of the cartridge 100 is in fluid communication with a first end of a corresponding one of fluid channels 17a-17d (see FIG. 8) in the second plane of fluid pathways of the cartridge 100. A second end of each of the fluid communication channels 17a-17d (see FIG. 8) are in fluid communication with a corresponding pressure port 22b, 22d, 22f, and 22h (see FIG. 8). The cartridge 100 couples with instrument via the pressure ports 22b, 22d, 22f, and 22h (see FIG. 8) to supply positive and negative pressure to facilitate mixing and testing of the sample as described below. FIGS. 6E and 6F also show a front perspective view and a back perspective view of FIGS. 5A, 5B, and 8 with additional labels corresponding to the description of this section.

### Sample Mixing

Referring to FIG. 5B unless indicated otherwise, each individual aliquot in the sample chambers 11 is pulled into a separate reservoir or channel (a serpentine channel pathway 13 (see FIG. 5A) in various embodiments) and brought into contact with the channel specific reagent, located in one (or both) of the two reagent pockets 14 (see FIG. 5A). Specifically, a pump in the instrument applies negative pressure to pressure ports 22b, 22d, 22f, and 22h (see FIG. 8) to draw the sample through the reagent pockets 14 (see FIG. 5A) and serpentine channel pathway 13 (see FIG. 5A). The reagents and sample are kept separate from each other during sample chamber 11 filling so as to avoid reagents floating on the blood and getting trapped in the filter in filter chambers 12, as well as to ensure fluidic isolation of the one or more sample chamber 11 from the heating chamber 6. To this end, precise time of when the test sample is in contact with the reagents can be measured thereby facilitating accurate and precise clot time measurements (e.g., from when mixing begins). In addition, the reagents and test samples are kept separated from one another, in some embodiments, until all channels are metered to prevent undesired siphoning of test samples from other channels or from the unprocessed sample in the fluidic pathways. The sample is aspirated through the serpentine channel 13 (see FIG. 5A) until it triggers an optical sensor in the instrument (detection zone is top of serpentine channel near the optical detection zone 15 (see FIG. 5A)) which closes off the channel from the pump. Mixing in the serpentine channels or zones, or region, therein can be controlled by one or more independent valves and pathways that allow individual channel control. Alternative sensor techniques could be utilized: pressure, pass through optical sensors, ultrasound detection, time, volumetric control, etc. Once all the channels' optical sensors trigger, the pump reverses and applies positive pressure to pressure ports 22b, 22d, 22f, and 22h (see FIG. 8). The positive pressure pushes the biological sample, such as blood, down the serpentine path 13 (see FIG. 5A) for a designated time, or until a second set of optical sensors in the instrument is tripped (in an alternative embodiment). This process, to pull the sample up the serpentine path 13 (see FIG. 5A) to the optical detection zone where it is detected by an optical sensor of the instrument and push back for a given time, repeats until full sample mixing is achieved. FIGS. 6E and 6F also show labels corresponding to the description of this section.

Other sensors (e.g., impedance sensors), pressure sensor, and etc., may be used. Alternatively, additional sensors may be used to detect both ends of the optical detection zone. Alternate pathway geometries, obstructions to create turbulence, cycle numbers, and cycle speed are all design alternatives that can be used with varying test types to achieve optimal results. In alternative embodiments, mixing could be achieved with one or more ferromagnetic beads or bars placed within the cartridge and controlled by the instrument.

### Test Chamber Filling

Referring to FIGS. 5A, 5B, and 8, one or more testing chambers 16 are filled after mixing is completed. Using one or more independent valves and pathways, each testing chamber 16 is filled with a sample via the application of pressure perturbations (negative and/or positive pressure) at pressure ports 22b, 22d, 22f, and 22h. Specifically, the instrument pump will apply negative pressure to pressure ports 22b, 22d, 22f, and 22h until all of the filters are clogged in the one or more filter chambers and cause a pressure spike to cause the instrument to turn the pump off, similar to filling the heating chamber 6. As discussed above, alternative filling detection techniques could be used. The testing chambers 16 have design features such as the ridges 24a which prevent the formation of bubbles in the testing chambers 16 during filling. Once filled, the instrument begins viscoelastic testing of the sample. FIGS. 6E and 6F also show labels corresponding to the description of this section.

In some embodiments, the cartridge apparatus includes, at least, four independent fluidic circuits configured with different sets of reagents for measurements (and/or sample preparation) to be performed in parallel. The measurements are performed per channel of the, at least, four channels of the cartridge. The measurement, in some embodiments, include viscoelastic properties such as a sample shear modulus. The measurement, in another embodiment, includes other properties such as viscosity, elastic modulus, or any other mechanical property of the sample, or combinations thereof.

Table 1 provides an example set of reagents and measurement parameters for use in an example cartridge apparatus (e.g., apparatus 100, among others). As shown in Table 1, Channel #1 in the example cartridge apparatus is interrogated to measure clot time of the test sample in the presence of kaolin, which is an activator of the intrinsic pathway of coagulation. As shown in Table 1, Channel #2 is interrogated to measure clot time of the test sample in the presence of kaolin and in further presence of heparinase I, which is a neutralizer of the anticoagulant heparin. As shown in Table 1, Channel #3 is interrogated to measure overall clot stiffness of the test sample in the presence of i) thromboplastin, which is an activator of the extrinsic pathway of coagulation, and ii) polybrene, which is a neutralizer of the anticoagulant heparin. As shown in Table 1, Channel #4 is interrogated to measure clot stiffness of the test sample with the same reagents as channel #3, but with the addition of abciximab (e.g., Clotinab® and/or ReoPro®), which is an inhibitor of platelet aggregation/contraction. As shown in Table 1, when the assay is configured to operate with citrated whole blood samples, calcium is added to all the reagent formulations.

TABLE 1
Reagents utilized in a preferred embodiment

| Channel # | Reagents | Measurement (units) |
|---|---|---|
| 1 | Kaolin, calcium, buffers | Clot time |
| | and stabilizers | (Seconds) |
| 2 | Kaolin, heparinase I, calcium, | Clot time |
| | buffers and stabilizers | (Seconds) |
| 3 | Thromboplastin, polybrene, | Clot stiffness |
| | calcium, buffers and stabilizers | (hecto Pascals) |
| 4 | Thromboplastin, polybrene, | Clot stiffness |
| | abciximab (and/or | (hecto Pascals) |
| | cytochalasin D), calcium, | |
| | buffers and stabilizers | |

Table 2 provides an additional example set of reagents and measurements for use in an example cartridge apparatus (e.g., apparatus 100, among others). As shown in Table 2, channel #2 includes an extrinsic pathway activator with inhibition of fibrinolysis by tranexamic acid (TXA). In addition to the measurements previously presented in Table 1, channels #2, channel #3, and channel #4 are interrogated to also measure clot stiffness changes, which, for example, can be related to the fibrinolytic process. In some embodiments, other channels can include reagents that inhibit fibrinolysis and can also be interrogated to measure clot stiffness changes. For example, channel #4 could also include TXA or other fibrinolysis inhibitor in order to measure clot stiffness in the absence of fibrinolysis.

TABLE 2
Reagents utilized in a preferred embodiment

| Channel # | Reagents | Measurement (units) |
|---|---|---|
| 1 | Kaolin, calcium, buffers | Clot time (Seconds) |
| | and stabilizers | |
| 2 | Thromboplastin, polybrene, | Clot stiffness |
| | calcium, tranexamic | (hectoPascals) and |
| | acid, buffers and stabilizers | Clot stiffness change |
| 3 | Thromboplastin, polybrene, | Clot stiffness (hecto |
| | calcium, buffers and stabilizers | Pascals) and clot |
| | | stiffness change |
| 4 | Thromboplastin, polybrene, | Clot stiffness |
| | abciximab (and/or | (hecto |
| | cytochalasin D), calcium, | Pascals) and clot |
| | buffers and stabilizers | stiffness change |

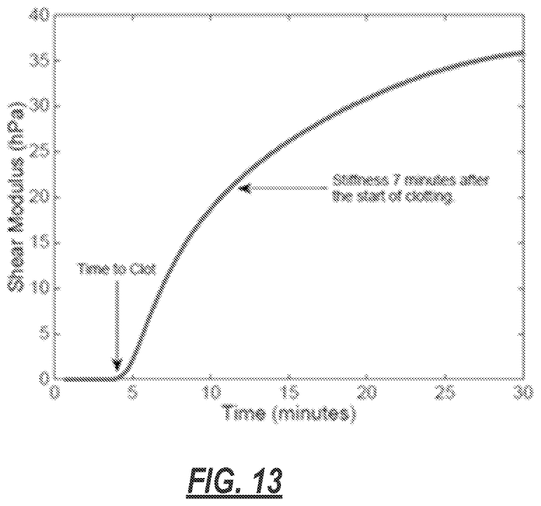

In some embodiments, clot time and clot stiffness are measured by analyzing a shear modulus (clot stiffness) versus time curve that is generated within each measurement channel in the cartridge. FIG. 13 shows an example shear module versus time curve, in accordance with an illustrative embodiment. Clot time may be determined by identifying when the clot stiffness meets or exceeds a threshold value, or when the first or higher derivative of such property being measured meets or exceeds a threshold value, or at the point of maximum acceleration in the rate of clot stiffness, or some combination of the above methods. Clot stiffness may be estimated by the clot stiffness at a fixed time after clot time, or the maximum overall clot stiffness measured within some time limit, or the clot stiffness at the point of maximum rate of change in clot stiffness, or some combination of the above methods. Similar methods can also be applied to measure the effects of fibrinolysis (i.e., clot dissolution) and the corresponding reduction in clot stiffness. In some embodiments, clot stiffness changes can be calculated as percentage drop in clot stiffness over a fixed time window, as a rate of change of clot stiffness over time, as area under or over the clot stiffness vs time curve within a predefined time window, as the time required to achieve a predefined drop in clot stiffness, or combinations thereof. Similar curves and similar measurements to those just described can be formed by plotting the Young's modulus, viscosity, or other viscoelastic property of the sample being measured.

TABLE 3
Parameters reported from measurement of the preferred
embodiments discussed in relation to Table 1.

| Hemostatic | | | |
|---|---|---|---|
| Index | Units | Description | Measurement |
| Clot Time | Minutes | Clot time | Clot time measured |
| | (min) | in citrated | from channel #1 |
| | | whole blood | with kaolin activation |
| | | | (intrinsic pathway) |
| Heparinase | Minutes | Clot time | Clot time measured |
| Clot Time | (min) | in citrated | from channel #2 |
| | | whole blood | with kaolin activation |
| | | with heparin | and heparinase I |
| | | neutralization | |
| Clot | hecto | Stiffness of | Clot stiffness |
| Stiffness | Pascals | the whole | measured from |
| | (hPa) | blood clot | channel #3 with |
| | | | thromboplastin |
| | | | activation (extrinsic |
| | | | pathway) and |
| | | | polybrene |
| Fibrinogen | hecto | Contribution of | Clot stiffness measured from |
| Contribution | Pascals | functional | channel#4 with thromboplastin |
| | (hPa) | fibrinogen | activation, polybrene, and |
| | | to clot stiffness | abciximab |
| Clot Time | Unit | Assessment of | Calculated ratio of |
| Ratio | less | residual heparin | clot time values |
| | | anticoagulation | from channels #1 and #2 |
| Platelet | hecto | Contribution of | Calculated from subtraction |
| Contribution | Pascals | platelet activity | of the clot stiffness values |
| | (hPa) | to clot stiffness | from channels #3 and #4 |

A person of ordinary skills in the art should recognize that clot time and clot stiffness can be estimated using a number of methodologies and criteria. Clot times and clot stiffness values obtained from the, at least, four channels/measurements may be combined to provide, at least, six parameters can depict a functional status of the patient's hemostatic system. The indexes are summarized in Table 3. Relationship between results (clot time, clot stiffness, clot stiffness change, etc.) from different channels may be verified to be within expected ranges as additional quality control checks to verify instrument, cartridge, and sample function.

In other embodiments, other reagents can be used and other hemostatic indexes or output parameters can be obtained such as in the case of a fibrinolytic index, indexes corresponding to the functionality of anti-platelet treatments, indexes corresponding to the functionality of anti-coagulation treatments, etc.

For example, one or more fibrinolysis indexes could be formed using the clot stiffness changes measured in any of the channels presented in Table 2, but preferably channels #3 and #4. Alternatively, a fibrinolysis index could be formed by differential combination of the clot stiffness changes measured in channels #2 and channel #3 presented in Table 2. Such combination could be in the form of a ratio, a difference, or combinations thereof. One of the benefit of using a combination of clot stiffness changes measured with and without an anti-fibrinoltyic reagent is the ability to mitigate the interfering effects of non-fibrinolysis driven reductions in clot stiffness values. In some embodiments, TXA or other fibrinolysis inhibitor reagent can be included in both channel #2 and channel #4 of the example cartridge of Table 2. With such modifications the parameters Clot Stiffness, Platelet Contribution, and Fibrinogen Contribution could be derived without the influence of fibrinolysis by combination of the clot stiffness measurements obtained in channel #2 and channel #4.

As discussed above, an example user interface is described in commonly assigned U.S. Pub. No. 2011/0252352 to Viola et al., which is incorporated by reference herein in its entirety. The example user interface may be used to display the measured hemostatic indexes as discussed in relation to Table 4, among other parameters.

TABLE 4
Parameters reported from measurement of the preferred
embodiments discussed in relation to Table 2.

| Hemostatic | | | |
|---|---|---|---|
| Index | Units | Description | Measurement |
| Clot Time | Minutes | Clot time in | Clot time measured |
| | (min) | citrated whole | from channel #1 |
| | | blood | with kaolin activation |
| | | | (intrinsic pathway) |
| Clot | hecto | Stiffness of the | Clot stiffness measured |
| Stiffness | Pascals | whole blood | from channel #3 with |
| | (hPa) | clot | thromboplastin activation |
| | | | (extrinsic pathway) |
| | | | and polybrene |
| Fibrinogen | hecto | Contribution of | Clot stiffness measured |
| Contribution | Pascals | functional | from channel #4 with |
| | (hPa) | fibrinogen | thromboplastin activation, |
| | | to clot | polybrene, and abciximab |
| | | stiffness | |
| Platelet | hecto | Contribution of | Calculated from subtraction |
| Contribution | Pascals | platelet activity | of the clot stiffness values |
| | (hPa) | to clot stiffness | from channels #3 and #4 |
| Clot | % or | Clot stiffness | Changes (% or rate of change) |
| Stiffness | hPa/sec | change over | in clot stiffness measured |
| Change | or sec | time | from channels #2 and #3 |
| Clot | % or | Differential rate | Differential comparison |
| Reduction | hPa/sec, | of clot stiffness | of clot stiffness change |
| Differential | or no | changes with | measured in channels |
| | units | and without | #2 and #3. |
| | | anti-fibrinolytic | |

As noted before, in various embodiments, the testing chambers 16 are shaped to facilitate ultrasound testing of viscoelastic properties of the sample, but alternative geometries can also be implemented to facilitate other types of testing. Such an ultrasound testing system is described in commonly assigned U.S. Pat. No. 9,726,647 and U.S. Pub. No. 2016/0139159, both of which are hereby incorporated by reference in their entirety. Ultrasound transducers in the measuring system connect with the testing chambers 16 of the cartridge 100 via compliant and deformable elastomers 21 which are affixed to a testing block 21d on the cartridge 100.

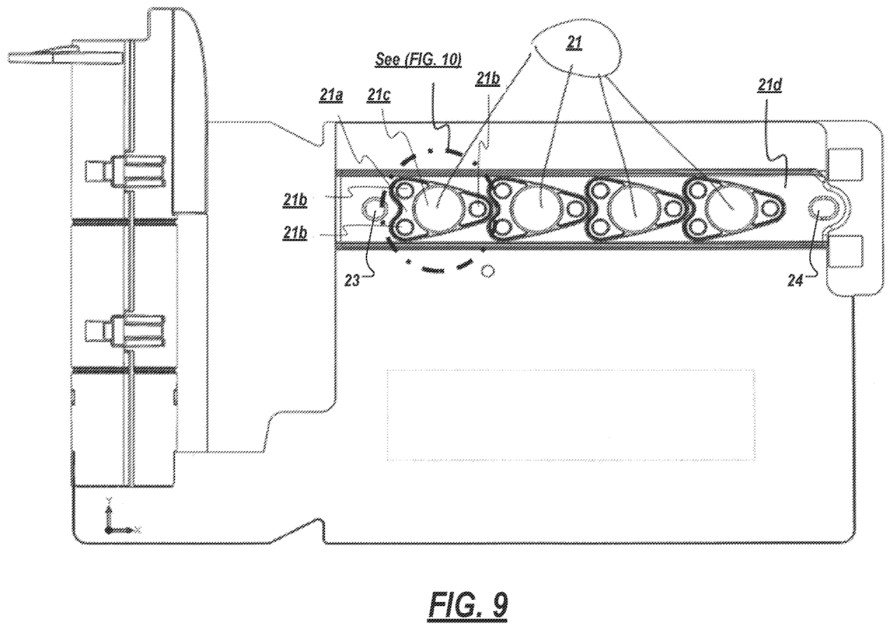

Example elastomeric materials optionally include, Dynaflex D3202, Versaflex OM 9-802CL, Maxelast 54740, RTP 6035, Versaflex CL2003X, among others. Referring now to FIG. 9 unless indicated otherwise, the testing block 21d is aligned with the testing chambers 16 (see FIG. 5B) via alignment slots 23 and 24 on the cartridge 100. Referring still to FIG. 9, the elastomers 21 may be affixed to the testing block 21d via a flange 21a on the elastomers 21. The flange 21a may have a plurality of alignment holes 21b that may receive corresponding alignment pegs (not shown) from the testing block 21d. The soft elastomers 21 may also each include a lens 21c that focuses ultrasound energy within the sample at the testing chambers 16.

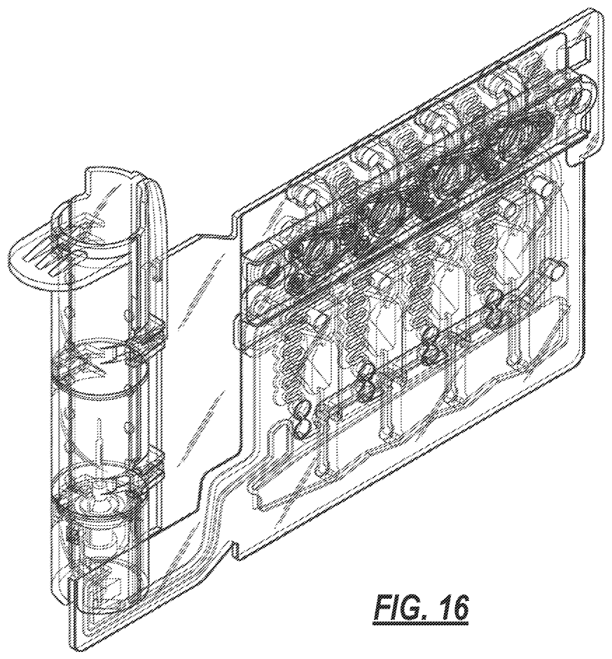

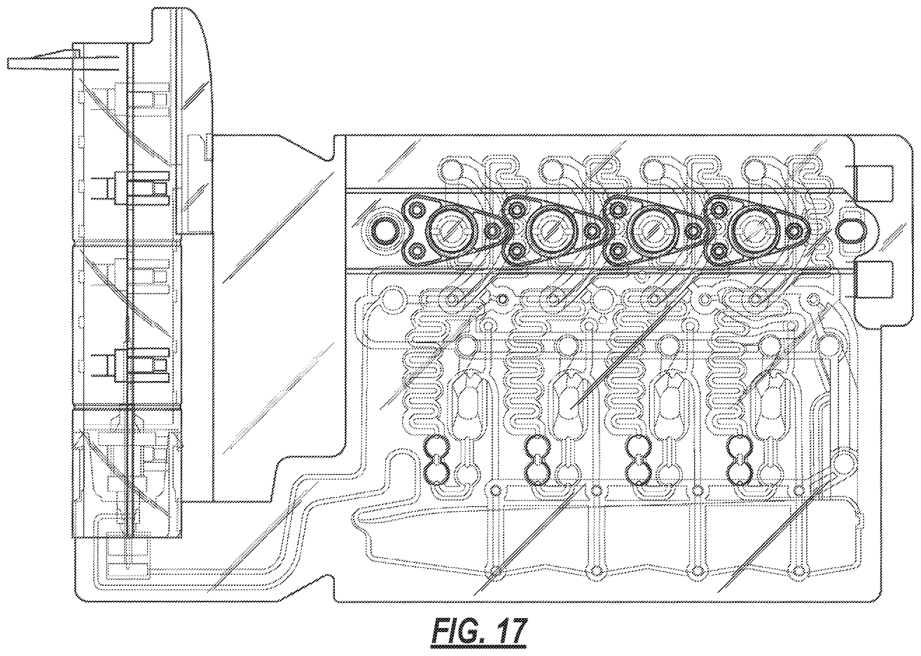

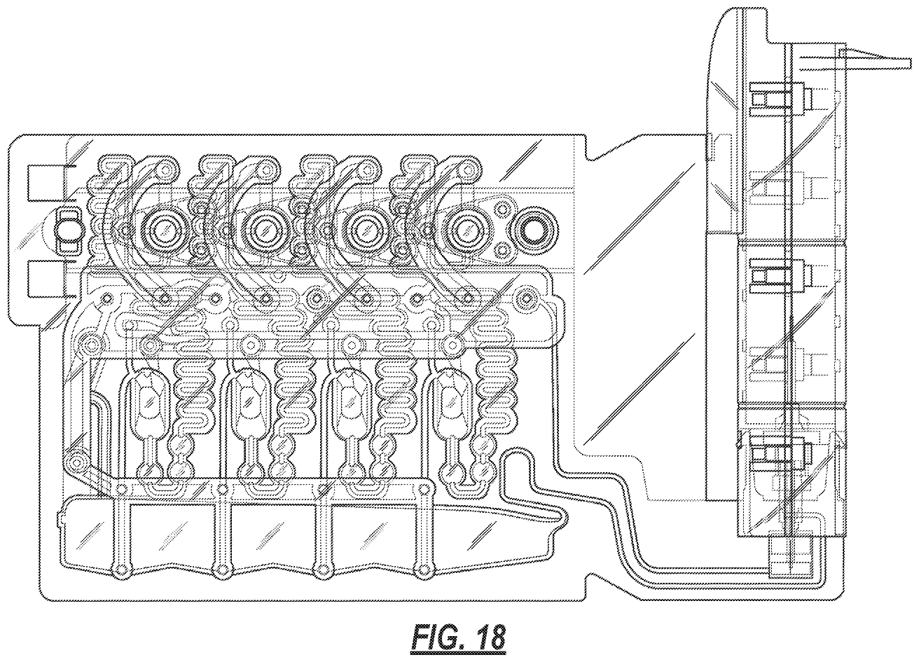

FIG. 16 shows a photograph of an exemplary cartridge of FIGS. 1, 2, 3, 4, 5A, 5B, 7, 8, and 9 for use in a disposable system, in accordance with an illustrative embodiment. FIG. 17 shows a front view of the exemplary cartridge of FIGS. 1, 2, 3, 4, 5A, 5B, 7, 8, and 9, in accordance with an illustrative embodiment. FIG. 18 shows a front view of the exemplary cartridge of FIGS. 1, 2, 3, 4, 5A, 5B, 7, 8, and 9, in accordance with an illustrative embodiments.

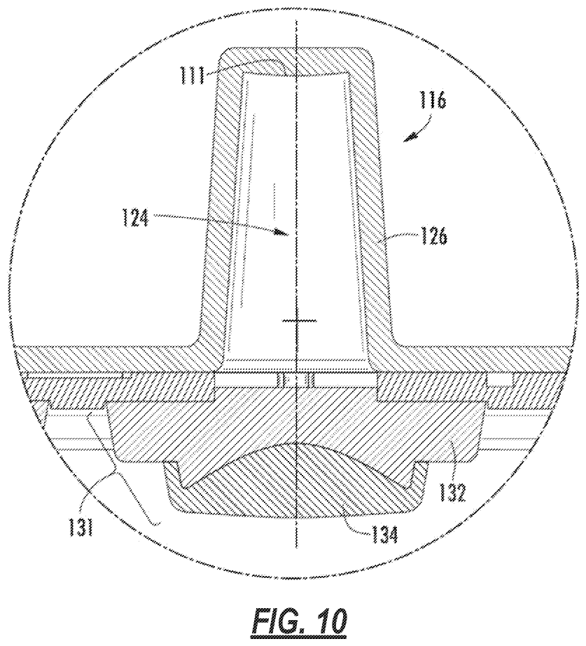

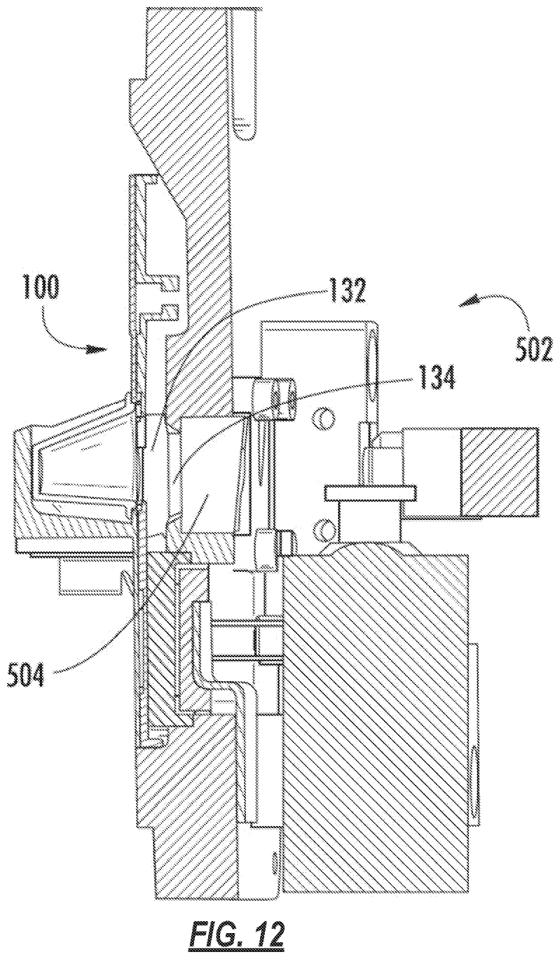

As described in U.S. Pat. No. 9,272,280, which is incorporated by reference herein in its entirety, in various embodiments, the consumable cartridge contains a lens assembly that focuses ultrasound energy within the sample that can be used to generate streaming and mixing. The lens assembly, or sound focusing assembly, is designed using a soft material, such as a thermoplastic elastomer 134 (previously referred to as 21), in conjunction with a rigid substrate 132 (e.g., formed of testing block 21d), such as polystyrene as shown in FIGS. 10, 11, and 12. This combination provides a dry ultrasound coupling that does not require the use of any fluid or gel couplant. Note that the same lens and ultrasound driver used for hemostasis measurement can be used in this matter to provide mixing. Increasing acoustic energy for mixing can be delivered by, for example, increasing pulse length, pulse amplitude or pulse repetition frequency.

Referring now to FIG. 10, a top cross-sectional view of the testing chamber 116 (referred to previously as testing chamber 16) is shown. To seal each test chamber, e.g. test chamber 116, a lens assembly 131 includes a rigid substrate 132 and a couplant 134 that can be positioned at the back end of each test chamber.

Referring still to FIG. 10, each couplant 134 comprises an elastomeric material. Optionally, the elastomeric material is a thermoplastic elastomer (TPE). Example elastomeric materials optionally include, Dynaflex D3202, Versaflex OM 9-802CL, Maxelast 54740, RTP 6035, Versaflex CL2003X, among others. Optionally the couplant is over-molded to the rigid substrate. Optionally the couplant is mechanically anchored to the rigid substrate.

Referring still to FIG. 10, between each couplant 134 and the open space of each test chamber is a rigid substrate 132. The rigid substrate and the couplant form an interface that focuses ultrasound transmitted (e.g. lens assembly) by an ultrasonic transducer into the chamber's open space and onto any biological fluid and/or reagents in the chamber. The rigid substrate of the lens can comprise a material which allows sound to pass and that can act to focus ultrasound at some level within the space. Optionally, the rigid substrate comprises a styrene.

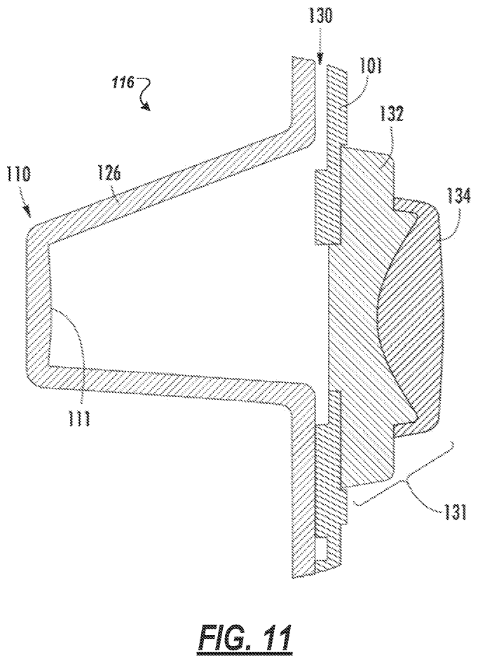

Referring now to FIG. 11, The lens assembly may be glued or welded to the surface 101 of the testing block 21d (shown in FIG. 11 as element 132) to secure the lens in place in an orientation that allows the desired focusing of sound. Alternatively, the lens assembly is optionally manufactured together with the surface 101 of the testing block 21d. In this regard, the rigid substrate 132 can be molded with the surface 101 of the testing block 21d and the couplant 134 can be overmolded or mechanically anchored on the rigid substrate. A wide variety of materials can be used to construct the device. For example, plastics can be used for single use, disposable cartridges.

Referring still to FIG. 11, each of the test chambers 116 can have a lens assembly positioned over the large opening of each chamber's open space. In this way, each chamber can be separately interrogated by focused ultrasound.

Referring still to FIG. 11, when placed in the instrument, the couplant 134 can be placed in acoustic communication with a transducer for supplying ultrasound through the lens assembly and into a test chamber 116. Optionally, an intermediate layer of an acoustically permeable material is positioned between an ultrasonic transducer and the couplant. For example, and intermediate layer or block of Rexolite® or TPX® can be used. The intermediate layer can be forced against the couplant and can be in acoustic contact with the transducer.

Referring still to FIG. 11, sound generated by a transducer passes through the intermediate layer, through the couplant, through the rigid substrate, and is focused within the biological sample, such as blood, and reagent in the test chamber. Some of the sound directed into chamber contacts the distal interior surface 111 of the test chamber, which is defined by the surface 126. Optionally, the surface is polystyrene. The distal interior surface has a known geometry and is positioned at a known distance from the ultrasound source. The distal interior surface 111 is used as a calibrated reflector, which is used to estimate the speed of sound and attenuation of sound in a test chamber at base line and during the process of clot formation and clot dissolution. These measurements can be used, for example, to estimate hematocrit of the subject along with the indexes of hemostasis. The sound generated by the transducer can be focused within the biological sample in a test chamber using a parabolic mirror that is coupled to the biological sample using an elastomer.

Other example cartridge apparatus and measurement system, and methods thereof, are described in U.S. Pat. No. 9,031,701; U.S. Provisional Appl. No. 61/443,084; U.S. Pat. Nos. 9,272,280; 9,410,971; U.S. Provisional Appl. No. 61/443,088; U.S. Publication No. 2011/0252352; published PCT Publication No. WO2011/127436; U.S. Publication No. 2012/0294767; U.S. Pat. Nos. 7,892,188; 8,740,818; and U.S. Publication No. 2016/0274067, each of which is incorporated by reference herein in its entirety.

As noted, the cartridge and features described herein can be modified for use with other types of measurement systems such as thromboelastography-based systems, thromboelastometry-based systems, optical-based systems, fluorescence-based systems, colorimetric-based systems, aggregometry-based systems, resonance-based system, and an electrical impedance-based system, among others.

Many modifications and other embodiments of the invention set forth herein will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used in the claims, the term “first”, “second”, and “third” are provided merely as labels and do not intended to connote a sequence.

1.-44. (canceled)

45. A method of mixing a sample with one or more reagents in an apparatus and testing a mixed sample for an assessment of hemostasis, the method comprising:
receiving a plurality of metered samples;
alternately and multiplicatively flowing at least one of the metered samples until the at least one metered sample is mixed with the one or more reagents, or a combination of reagents, to form a mixed metered sample, wherein the at least one metered sample is alternately and cyclically flowed i) in a first direction from a first metering channel through one or more reagent pockets, with the one or more reagents therein, and along a serpentine pathway in communication with the first metering channel until at least a portion of the at least one metered sample reaches a detection zone located in, or after, the serpentine pathway and ii) in a second direction from the detection zone reserved to the first direction through at least a portion of the serpentine pathway toward the first metering channel; and
driving the mixed metered sample in a testing chamber in fluidic communication with the first metering channel, wherein the testing chamber is structurally configured for interrogation by a measurement system configured to determine properties of the mixed metered sample, and wherein an interrogation of the testing chamber is performed by the measurement system with the mixed metered sample located therein.

46. The method of claim 45, further comprising:
receiving the test fluid in a first chamber configured to substantially adjust a temperature of the test sample towards a body temperature, wherein the metered sample received in the first metering channel is received from the first chamber.

47. The method of claim 45, wherein the test fluid is moved into the first chamber in response to a first applied positive or negative pressure that is applied by, or generated from, the measurement system.

48. The method of claim 45, further comprising:
conditioning the test fluid in the first chamber to, or substantially near, a desired temperature, wherein the test fluid is mixed with the one or more reagents following exit from the first chamber.

49. The method of claim 45, further comprising:
isolating the plurality of metered samples in a plurality of metering channels to prevent the plurality of metered samples from contacting the one or more reagents during filling of the plurality of metering channels.

50. The method of claim 45, wherein the first applied positive or negative pressure is applied by, or generated from, the measurement system in reversed so as to move the at least one metered sample in the second direction; and
wherein a second applied positive or negative pressure is applied by, or generated from, the measurement system at a second port in communication with the serpentine pathway so as to move the at least one metered sample in the second direction.

51. The method of claim 45, wherein the receiving the mixed metered sample in the testing chamber further comprises:
receiving a negative or differential pressure via a third pressure port, wherein the third pressure port is further in fluid communication with the testing chamber.

52. The method of claim 51, wherein the testing chamber is downstream of the serpentine pathway and the third pressure port is downstream of the testing chamber.

53. The method of claim 45, wherein a first set of one or more reagent pockets includes a first set of one or more reagents, or the combination of reagents, wherein the first set of one or more reagents, or the combination of reagents, is in the form of one or more lyophilized beads.

54. The method of claim 53, wherein the first set of one or more reagent pockets associated with the first testing chamber comprises kaolin, calcium, buffers, and stabilizers, wherein a first testing chamber forms a part of a first test channel of the cartridge.

55. The method of claim 53, wherein a second set of one or more reagent pockets associated with a second test chamber comprises: a combination of kaolin, heparinase I, calcium, buffers and stabilizers; or a combination of thromboplastin, polybrene, tranexamic acid, calcium buffers, and stabilizers, wherein the second testing chamber forms a part of a second test channel of the cartridge.

56. The method of claim 53, wherein a third set of one or more reagent pockets associated with a third test chamber comprises: thromboplastin, polybrene, calcium, buffers, and stabilizers, wherein the third testing chamber forms a part of a third test channel of the cartridge, and wherein a fourth set of one or more reagent pockets associated with a fourth test chamber comprises: thromboplastin, polybrene, at least one of abciximab and cytochalasin D, calcium, buffers, and stabilizers, wherein the fourth testing chamber forms a part of a fourth test channel of the cartridge.

57. The method of claim 45, wherein the measurement system is configured to determine at least one of:
a clot time, a clot stiffness change, and/or a clot reduction differential of the first mixed sample, and
a clot stiffness or a heparinase clot time of a second mixed sample.

58. The method of claim 45, wherein the measurement system is configured to determine shear modulus of the first mixed sample as the determined one or more viscoelastic properties of one or more of the first mixed sample, the second mixed sample, the third mixed sample, or combinations thereof.

59. The method of claim 45, wherein the first set of one or more reagent pockets associated with the first testing chamber comprises a coagulation activator selected from the group consisting of kaolin, Hageman factor, celite, glass, ellagic acid, micronized silica, tissue factor, recombinant tissue factor, thromboplastin, thrombin, factor Xa, reptilase, ecarin, and Russell's viper venom.

60. The method of claim 45, wherein a second set of one or more reagent pockets associated with a second test chamber comprises a platelet activator or inhibitor selected from the group consisting of abciximab, eptifibatide, tirofiban, roxifiban, orbofiban, cytochalasin D, blebbistatin, PAR1 inhibitors, PAR4 inhibitors, glycoprotein I.B. inhibitors, thrombin receptor activating peptide (TRAP), adenosine diphosphate (A.D.P.), and arachidonic acid.

61. The method of claim 45, wherein a third set of one or more reagent pockets associated with a third test chamber comprises a fibrinolytic function activator or inhibitor selected from the group consisting of tPA, uKA, streptokinase, TAFIa, plasmin/plasminogen, aprotinin, epsilon-aminocaproic acid, tranexamic acid, plasminogen activator inhibitor 1 (PAI1), α2-antiplasmin (α2-AP), plasmin-antiplasmin complexes, and carboxypeptidase inhibitor.

62. The method of claim 45, wherein a fourth set of one or more reagent pockets associated with a fourth test chamber comprises a reagent selected from the group consisting of FXIIIa inhibitor, Hexadimethrine bromide (polybrene), heparinase, ristocetin, heparin, low molecular weight heparin, corn trypsin inhibitor, adenosine, GPRP, calcium, fibronectin, collagen, epinephrine, immuno-detection reagent, direct thrombin inhibitor, factor Xa inhibitor, direct thrombin inhibitor, and thrombomodulin.

63. The method of claim 45, wherein the measurement system is selected from a group consisting of a sonorheometry-based system, thromboelastography-based system, a thromboelastometry-based system, an optical-based system, a fluorescence-based system, a colorimetric-based system, an aggregometry-based system, a resonance-based system, and an electrical impedance-based system.

64. An apparatus configured to perform the method of claim 45.
